(19) United States
(12) Patent Application Publication
(10) Pub. No.: US 2024/0369570 A1
(43) Pub. Date: Nov. 7, 2024

(54) OFF THE SHELF PROXIMITY BIOTINYLATION ENZYME

(71) Applicant: Stichting Radboud Universiteit, Nijmegen (NL)

(72) Inventors: Michiel Vermeulen, Nijmegen (NL); Guido VAN MIERLO, Nijmegen (NL); Irene SANTOS-BARRIOPEDRO, Nijmegen (NL)

(21) Appl. No.: 18/285,734

(22) PCT Filed: Apr. 8, 2022
(86) PCT No.: PCT/NL2022/050197

§ 371 Date: Oct. 5, 2023

(30) Foreign Application Priority Data
Apr. 9, 2021 (EP) 21167725.7

(51) Int. Cl. G01N 33/68; C07K 14/31; C07K 14/315; C07K 16/12; C07K 16/18; C07K 16/40; C12N 9/00; G01N 33/573

(52) U.S. Cl. CPC G01N 33/6875; C07K 14/31; C07K 14/315; C07K 16/1275; C07K 16/18; C07K 16/40; C12N 9/93; C12Y 603/04015; G01N 33/573; C07K 2319/70; C07K 2319/705; G01N 2333/914

(57) ABSTRACT

The present invention provides a fusion polypeptide comprising a biotin ligase enzyme fused to an immunoglobulin-binding bacterial protein, preferably wherein the immunoglobulin-binding bacterial protein is selected from Protein A, Protein G, Protein A/G and Protein L. The fusion polypeptide is preferably provided in combination with an antibody to which the immunoglobulin-binding bacterial protein can bind, and which combination is used for targeted proximity biotinylation.

### FIELD

The invention is in the field of protein labelling and detection, and in particular relates to an enzyme that facilitates proximity biotinylation experiments in primary cells and can be used to understand how proteins cooperate in vivo and how this contributes to cellular homeostasis and disease

### INTRODUCTION

Proximity biotinylation recently emerged as a powerful interaction proteomics technology that can be used to identify direct and indirect interactions between proteins in vivo¹⁻³. This technology typically involves fusing a proximity biotinylation enzyme to target proteins of interest using CRISPR-based knock-in strategies or plasmid-based expression. Upon addition of exogenous biotin, proteins that are in close proximity to the bait protein during the biotin pulse become biotinylated. These biotinylated proteins can subsequently be enriched from crude cell lysates using streptavidin conjugated beads and analysed by quantitative mass spectrometry. Various proximity biotinylation enzymes have been described, including BioID, BioID2, APEX and TurboID⁴. TurboID in particular is a very attractive proximity biotinylation enzyme since it is a very fast enzyme, which labels bait-proximal proteins in minutes. Furthermore, unlike the APEX enzyme which relies on H₂O₂ for its enzymatic activity, TurboID based proximity biotinylation only requires exogenous addition of biotin to target cells and is therefore not toxic for target cells.

Proximity biotinylation enzymes have been used for various biological questions, for example for temporal profiling of DNA damage response pathways, to decipher cellular signalling pathways and for organelle-specific proteome profiling in cell culture cells and model organisms⁵⁻⁸. However, as mentioned above, these approaches typically rely on CRISPR-based knock-in or plasmid-based expression approaches to introduce a biotinylation enzyme fused to a bait protein in target cells of interest. This is not only labor intensive but also restricts proximity biotinylation technology to cells that can be genetically engineered and maintained and propagated for a long period of time in vitro. There is therefore a need for technology to overcome this bottleneck and that facilitates proximity biotinylation workflows in primary cells in the absence of genetic engineering or transfection.

### SUMMARY OF THE INVENTION

Here we present a new recombinant proximity biotinylation enzyme, called ProtA-Turbo, which consists of Protein A fused to the TurboID proximity biotinylation enzyme. Upon target cell permeabilization using either fixed or non-fixed mammalian cells, the ProtA-Turbo enzyme can be targeted to baits of interest using antibodies against endogenous proteins or protein modifications. Bait proximal proteins are subsequently biotinylated upon addition of exogenous biotin. Cells are then lysed using high stringency lysis and biotinylated proteins are affinity enriched in triplicate using streptavidin-conjugated beads using appropriate negative controls. Data visualisation reveals statistically significant in vivo bait-proximal proteins. To benchmark this method, we combined the ProtA-Turbo enzyme with antibodies against various well-characterized baits: Emerin, which resides in the nuclear envelope, the heterochromatin modification H3K9me3 and a chromatin remodeler protein BRG1, which is part of the Swi/Snf complex, in various cell types. For all these baits, confocal microscopy revealed that the ProtA-Turbo enzyme and associated biotinylation is targeted to appropriate regions in mammalian nuclei. Affinity purifications and label free quantitative mass spectrometry revealed numerous positive controls as well as new proximal proteins for all the used baits. Finally, follow up experiments revealed that FLYWCH1 is a novel H3K9me3 associated protein that interacts with H3K9me3-marked centromeric heterochromatin. Primary validated antibodies that have been used to target the ProtA-Turbo moiety inside cells include, but are not limited to, the post-translational modifications H3K9me3, H3K4me3 and H3K27ac, and the proteins Emerin, BRG1, CENPC and INCENP.

In summary, the recombinant ProtA-Turbo enzyme represents a new ‘off the shelf’ proximity biotinylation enzyme that can be used for in vivo interaction proteomics studies in fixed and non-fixed primary cells or clinical samples.

In a first aspect, the present invention provides a fusion polypeptide comprising a biotin ligase enzyme fused to an immunoglobulin-binding bacterial protein, preferably wherein the immunoglobulin-binding bacterial protein is selected from Protein A, Protein G, Protein A/G and Protein L.

In a preferred embodiment of a fusion polypeptide according to the invention, the biotin ligase enzyme has proximity-dependent biotinylation activity. Hence, the biotin ligase enzyme is capable of proximity-dependent biotinylation of proteins.

In a further aspect, the present invention provides a composition or combination comprising the fusion polypeptide as described above, and further comprising an immunoglobulin, preferably an antibody, such as a polyclonal antibody or monoclonal antibody, more preferably a monoclonal antibody, wherein said antibody targets the fusion polypeptide to a subcellular region of interest.

In a further aspect, the present invention provides a complex comprising the fusion polypeptide according to the invention as described above complexed to an immunoglobulin, preferably an antibody, such as a polyclonal antibody or monoclonal antibody, more preferably a monoclonal antibody, wherein said antibody targets the complexed fusion polypeptide to a subcellular region or protein of interest.

In a preferred embodiment of a composition according to the invention or a complex according to the invention, the antibody is an IgG antibody, preferably the immunoglobulin-binding bacterial protein binds to the Fc region of said IgG.

The present invention provides a fusion polypeptide comprising a biotin ligase enzyme fused to an immunoglobulin-binding bacterial protein, preferably wherein the immunoglobulin-binding bacterial protein is selected from Protein A, Protein G, Protein A/G and Protein L. The fusion polypeptide is preferably provided in combination with an antibody to which the immunoglobulin-binding bacterial protein can bind, and which combination is used for targeted proximity biotinylation.

In another aspect, the present invention provides a method for biotinylating a protein of interest in a cell, a subcellular region or a sample of interest, the method comprising:
a) contacting the sample with the composition or the complex of the invention as described above; and b) adding biotin or a derivative thereof and ATP to the sample, wherein the biotin ligase biotinylates the protein.

In another aspect, the present invention provides a method of proximity labeling of proteins in a cell, the method comprising:
a) introducing the complex or composition of the invention as described above into a cell, wherein the fusion polypeptide comprising the biotin ligase is targeted to a subcellular region of interest; and b) contacting the cell with biotin or a derivative thereof and ATP, wherein proteins in proximity to the biotin ligase are biotinylated.

In another aspect, the present invention provides a kit of part for biotinylating a protein of interest in a cell, a subcellular region or a sample of interest, comprising a fusion polypeptide according to the invention as described above, and an immunoglobulin, preferably an antibody, more preferably a monoclonal antibody, preferably an antibody to which the immunoglobulin-binding bacterial protein binds. An antibody may include a part of an antibody to which the immunoglobulin-binding bacterial protein binds. In a preferred embodiment of a kit of the invention, the immunoglobulin targets the fusion polypeptide to a protein or subcellular region of interest. This targeting is preferably the result of binding of the immunoglobulin to a protein of a cell, and the prior, simultaneous or subsequent binding of the fusion polypeptide of the invention to the immunoglobulin. In a further preferred embodiment of a kit of the invention, the kit further comprising biotin, or a derivative thereof, and ATP. A biotin derivative is preferably a labelling compound comprising a biotin or biotinyl moiety reactive with biotin ligase enzyme of the fusion protein.

### BRIEF DESCRIPTION OF THE DRAWINGS

FIG. 1: A ProteinA-TurboID fusion protein allows enrichment of protein localized to specific sub-nuclear compartment in crosslinked cells. (a) Schematic outline of the method. (b) Immunofluorescence images of targeting the nuclear lamina (via Emerin), the SWI/SNF protein complex (via BRG1) or (pericentromeric) heterochromatin (via H3K9me3) in crosslinked HeLa cells. IgG was used as a control antibody. Biotinylation (in green) overlapping the antibody signal (in red) illustrates correct localization of the ProtA-Turbo fusion protein. Scale bars represent 10 μm. (c) Immunoprecipitation of biotinylated proteins after targeting (T) proteins as in (b). IgG was used as a control (C). (d) Volcano plot of mass spectrometry analyses of biotin IPs as in (c). A selection of proteins known to localize to the targeted proteins are highlighted. In the H3K9me3 volcano plot, red dots indicate writer/writer complexes and blue indicates known pericentromeric proteins. Protein names in white indicate common streptavidin contaminants (e,f) Biotin ChIP-seq after H3K9me3 or IgG targeting with ProtA-Turbo.

FIG. 2: A ProteinA-TurboID fusion protein allows enrichment of protein localized to specific sub-nuclear compartment in non-crosslinked cells. (a) Schematic outline of the method. (b) Immunofluorescence images of targeting the nuclear lamina (via Emerin), the SWI/SNF protein complex (via BRG1) or (pericentromic) heterochromatin (via H3K9me3) in non-crosslinked HeLa cells. IgG was used as control antibody. Biotinylation (in green) overlapping the antibody signal (in red) illustrates correct localization of the ProtA-Turbo fusion protein. Scale bars represent 10 μm. (c) Immunoprecipitation of biotinylated proteins after targeting (T) proteins as in (b). IgG was used a control (C). (d) Volcano plot of mass spectrometry analyses of biotin IPs as in (c). A selection of proteins known to localize to the targeted proteins are highlighted. In the H3K9me3 volcano plot, red dots indicate writer/writer complexes and blue indicates known pericentromeric proteins. Protein names in white indicate common streptavidin contaminants.

FIG. 3: ProtA-Turbo targeting of H3K9me3 reveals FLYWCH1 as a protein localized to (peri) centromeric heterochromatin. (a) Immunofluorescence of FLYWCH1 antibody staining (left), FLYWCH1-GFP overexpression (middle) or GFP-signal of FLYWCH1 endogenously tagged with GFP (right). The large panel is the merge, and the small panels are H3K9me3 (red), antibody/GFP signal (green) or DAPI (blue). (b) Representative screenshot of a FLYWCH1-bound genomic regions as assessed by ChIP-seq. IgG was used as control antibody. Grey markers indicate different repeat types from the Repeatmasker. (c) Most enriched DNA motifs under 451 FLYWCH1-specific peaks. (d) Intersection of FLYWCH1 ChIP-seq reads with most enriched repeat types/sequences that are at least 10-fold differential between FLYWCH1 and IgG ChIP-seq. (e) Immunofluorescence analyses of FLYWCH1 fused to miniTurboID. FLYWCH1 is visualized by staining the V5 tag (red). Scale bars represent 10 μm. (f) Volcano plot of mass spectrometry analyses of biotin IPs of FLYWCH1-miniTurboID with biotin compared to control (without biotin). Protein names in white indicate common streptavidin contaminants.

FIG. 4: Map of pK19 Protein A Turbo plasmid (pK19-6xHis-ProtA Turbocomplete) (a) Overview. (b) Linker sequences.

### DETAILED DESCRIPTION OF THE INVENTION

The present invention provides a new enzyme, called ProtA-Turbo, which can be used for proximity biotinylation and interaction proteomics purposes without the requirement for genetic manipulation or transfection of target cells. We illustrate the applicability of the ProtA-Turbo enzyme by targeting three nuclear baits using polyclonal antibodies and we show that the enzyme can be used for interaction proteomics studies using fixed and non-fixed cells. Finally, we illustrate the usefulness of the ProtA-Turbo enzyme through the identification and initial characterization of a relatively uncharacterized protein, FLYWCH1, as a novel marker of centromeric, H3K9me3-marked chromatin.

Over the years, a range of methods have been developed to determine the proximal proteome of a protein of interest. ChIP-MS based approaches (i.e. antibody-based enrichment of chromatin fragments) are solely applicable to chromatin-bound proteins, require sonication, which is a highly variable process, and take several days to perform, thus limiting throughput⁶. Proximity biotinylation requires transfection or genetic manipulation, which potentially induces biological changes in the cell type of interest. Furthermore, genetic manipulation of target cells is labor intensive and is not applicable to all types of cells, such as non-proliferative primary cells. As an alternative, antibody-HRP (horseradish peroxidase) conjugates can be used to biotinylate local environments, but these methods are restricted to fixed material since HRP is denatured in the acidic environment of live cells^{15,16}. The fixed version of the off-the-shelf method presented here extends on the principle of the antibody-HRP conjugates, achieves higher enrichment of target-site specific GO terms and is faster to perform.

While off-the-shelf proximity biotinylation on cross-linked cells enriches the local proteome of a protein of interest, the usage of formaldehyde-based crosslinking precludes certain downstream applications. A notable example comprises crosslinking-mass spec (XL-MS), which can be used to discriminate direct from indirect protein-protein interactions¹⁷. This approach almost exclusively targets unmodified lysine residues¹⁸, but formaldehyde fixation of cells also targets these lysines. To allow future studies aimed at combining XL-MS with proximity biotinylation, omission of crosslinking would be beneficial. The native off-the-shelf workflow developed in the current study thus allows additional downstream workflows, including assessing protein-protein interaction topologies using XL-MS. In addition, we noticed that some cell types, such as U937 cells, have a tendency to clump after crosslinking, resulting in loss of material. The native workflow circumvented these issues and allowed to obtain a comprehensive H3K9me3 proximal proteome in U937 cells. Another advantage of the native version is that it is faster to perform compared to the fixed protocol. While the crosslinking-based and native workflow yield comparable results, the choice of method will depend on potential downstream applications, target cells of interest and available lab infrastructure.

The present inventors focused on targeting the ProtA-Turbo enzyme to nuclear proteins and a transcriptionally repressive histone modification. Future applications of the ProtA-Turbo enzyme will include targeting cytoplasmic and cell-surface proteins, for example in the context of cancer immunotherapy.

Furthermore, modifications on nucleic acids such as DNA or RNA methylation can be targeted using commercially available high-quality antibodies against these modifications. Other options include fusing TurboID to specific chromatin reader domains to generate an off-the-shelf alternative to the recently developed ChromID technology¹⁹. Together, these off the shelf approaches provide a highly flexible toolbox to perform proximity biotinylation assays in any cell type of interest in a fast and efficient manner.

The biotin ligase enzyme in fusion polypeptides in some preferred embodiments of this invention may be a wild type biotin ligase or it may be a modified biotin ligase. The biotin ligase in aspects of this invention may be a promiscuous biotin ligase enzyme, preferably, the biotin ligase is a promiscuous biotin ligase enzyme.

In preferred embodiments, the biotin ligase enzyme may be an engineered promiscuous biotin ligase enzyme as described in WO2019143529. Reference to such a biotin ligase enzyme is intended by the term “modified biotin ligase enzyme”. For instance, the biotin ligase may have at least one mutation comprising an amino acid substitution selected from the group consisting of Q65P, M209V, V160A, S150G, L151P, I305V, I87V, R118S, T192A, K194I, E140K, Q141R, M241T, and S263P, wherein positions of the amino acids are numbered relative to the reference wild-type biotin ligase sequence here below.

The wild-type biotin ligase sequence is the following:

```
Met Lys Asp Asn Thr Val Pro Leu Lys Leu Ile Ala Leu Leu Ala Asn
1 5 10 15
Gly Glu Phe His Ser Gly Glu Gln Leu Gly Glu Thr Leu Gly Met Ser
                        20 25 30
Arg Ala Ala Ile Asn Lys His Ile Gln Thr Leu Arg Asp Trp Gly Val
                35 40 45
Asp Val Phe Thr Val Pro Gly Lys Gly Tyr Ser Leu Pro Glu Pro Ile
        50 55 60
Gln Leu Leu Asn Ala Lys Gln Ile Leu Gly Gln Leu Asp Gly Gly Ser
65 70 75 80
Val Ala Val Leu Pro Val Ile Asp Ser Thr Asn Gln Tyr Leu Leu Asp
                                85 90 95
Arg Ile Gly Glu Leu Lys Ser Gly Asp Ala Cys Val Ala Glu Tyr Gln
                        100 105 110
Gln Ala Gly Arg Gly Arg Arg Gly Arg Lys Trp Phe Ser Pro Phe Gly
                115 120 125
Ala Asn Leu Tyr Leu Ser Met Phe Trp Arg Leu Glu Gln Gly Pro Ala
        130 135 140
Ala Ala Ile Gly Leu Ser Leu Val Ile Gly Ile Val Met Ala Glu Val
145 150 155 160
Leu Arg Lys Leu Gly Ala Asp Lys Val Arg Val Lys Trp Pro Asn Asp
                                165 170 175
Leu Tyr Leu Gln Asp Arg Lys Leu Ala Gly Ile Leu Val Glu Leu Thr
                        180 185 190
Gly Lys Thr Gly Asp Ala Ala Gln Ile Val Ile Gly Ala Gly Ile Asn
                195 200 205
Met Ala Met Arg Arg Val Glu Glu Ser Val Val Asn Gln Gly Trp Ile
        210 215 220
Thr Leu Gln Glu Ala Gly Ile Asn Leu Asp Arg Asn Thr Leu Ala Ala
225 230 235 240
Met Leu Ile Arg Glu Leu Arg Ala Ala Leu Glu Leu Phe Glu Gln Glu
                                245 250 255
Gly Leu Ala Pro Tyr Leu Ser Arg Trp Glu Lys Leu Asp Asn Phe Ile
                        260 265 270
Asn Arg Pro Val Lys Leu Ile Ile Gly Asp Lys Glu Ile Phe Gly Ile
                275 280 285
Ser Arg Gly Ile Asp Lys Gln Gly Ala Leu Leu Leu Glu Gln Asp Gly
        290 295 300
Ile Ile Lys Pro Trp Met Gly Gly Glu Ile Ser Leu Arg Ser Ala Glu
305 310 315 320
Lys
```

The (modified) biotin ligase enzyme in fusion polypeptides in some preferred embodiments of this invention have proximity-dependent biotinylation activity. Proximity-dependent biotinylation (PDB) activity can be tested using methods well known in the art, or as described herein, using e.g. miniTurbo and TurboID sequences as reference enzymes for such activity. In PDB, the biotin ligase catalyzes the covalent transfer of biotin (or other derivatives) to endogenous proteins that are located within a certain distance of the enzyme. By fusing the enzyme to specific proteins (referred to as “baits”), the enzyme can be localized to distinct areas of the cell, for example to a protein complex or an organelle. Addition of the enzyme substrate leads to the covalent biotinylation of proteins located near the bait (these are referred to as “preys”). Importantly, the labeling can be performed in live cells (or whole organisms), on fixed samples, or even in lysates or semi-purified structures. The primary advantage of PDB is that protein-protein interactions or the integrity of organelles do not need to be maintained post-labeling as the covalently biotinylated preys can be captured using an affinity matrix, most often streptavidin.

The (modified) biotin ligase enzyme in fusion polypeptides in some preferred embodiments of this invention may comprise an amino acid sequence having at least 90% sequence identity to the wild-type biotin ligase sequence, wherein the biotin ligase is capable of proximity-dependent biotinylation of proteins. Percent identity refers to % sequence identity over the full length of the sequence.

The (modified) biotin ligase may optionally comprise an N-terminal deletion of at least one amino acid up to the first 63 amino acids as numbered relative to the reference wild-type biotin ligase sequence described herein.

The (modified) biotin ligase may alternatively comprise an N-terminal deletion of the first 63 amino acids (D(1-63)) as numbered relative to the reference wild-type biotin ligase sequence described herein.

In other preferred embodiments, the modified biotin ligase enzyme may comprise the following amino acid substitutions:
a) Q65P, R118S, L151P, 1305V, and E313K amino acid substitutions,
b) R118S and E313K amino acid substitutions,
c) Q65P, R118S, L151P, 1305V, and E313R amino acid substitutions,
d) R118S and E313R amino acid substitutions,
e) R118S, L151P, and I305V amino acid substitutions,
f) K2E, R118S, M157T, and L298P amino acid substitutions,
g) R118S and L297P amino acid substitutions,
h) R118S, 1313N amino acid substitutions,
i) R118S, L151P, and I305V amino acid substitutions,
j) Q65P, R118S, and I305V amino acid substitutions,
k) Q65P, R118S, and L151P amino acid substitutions,
l) R118S, L151P, I305V, and K313R amino acid substitutions,
m) Q65P, R118S, I305V, and K313R amino acid substitutions,
n) Q65P, R118S, and K313R amino acid substitutions,
o) R118S, L151P, and K313R amino acid substitutions,
p) R118S, 1305V, and K313R amino acid substitutions,
q) Q65P and R118S amino acid substitutions,
r) R118S and L151P amino acid substitutions,
s) R118S and I305V amino acid substitutions,
t) R118S and M157T amino acid substitutions,
u) R118S and L298P amino acid substitutions,
v) K2E, R33G, R118S, M157T, and L298P amino acid substitutions,
w) K2E, R118S, M157T, I279T, L298P, and K307N amino acid substitutions,
x) Q65P, R118S, L151P, I305V, Y111H, and R118S amino acid substitutions,
y) Q65P, R118S, S150G, L151P, T192A, I305V amino acid substitutions,
z) Q65P, R118S, L151P, I231V, and I305V amino acid substitutions,
aa) Q65P, R118S, L151P, T192A, and I305V amino acid substitutions,
bb) Q65P, R118S, S150G, L151P, and I305V amino acid substitutions,
cc) R33G, Q65P, R118S, S150G, L151P, T192A, and I305V amino acid substitutions,
dd) N37S, Q65P, R118S, S150G, L151P, T192V, E266L, and I305V amino acid substitutions,
ee) Q65P, R118S, S150G, L151P, T192A, I280V, I305V, and A318V amino acid substitutions,
ff) Q65P, R118S, Q142R, S150G, L151P, T192A, M209V, and I305V amino acid substitutions,
gg) Q65P, R118S, S150G, L151P, T192A, and I305V amino acid substitutions,
hh) Q65P, R118S, Q142R, S150G, L151P, T192A, and I305V amino acid substitutions,
ii) Q65P, R118S, S150G, L151P, T192A, M209V, and I305V amino acid substitutions,
jj) Q65P, 187V, R118S, E141K, S150G, L151P, V160A, T192A, M209V, and I305V amino acid substitutions,
kk) Q65P, 187V, R118S, E141K, S150G, L151P, T192A, and I305V amino acid substitutions,
ll) Q65P, R118S, S150G, L151P, V160A, T192A, M209V, and I305V amino acid substitutions,
mm) Q65P, R118S, Q142R, S150G, L151P, V160A, T192A, M209V, and I305V amino acid substitutions,
nn) Q65P, 187V, R118S, E141K, S150G, L151P, V160A, T192A, M209V, and I305V amino acid substitutions,
oo) Q65P, I87V, R118S, Q142R, S150G, L151P, T192A, M209V, and I305V amino acid substitutions,
pp) Q65P, R118S, E141K, Q142R, S150G, L151P, T192A, M209V, and I305V amino acid substitutions,
qq) Q65P, 187V, R118S, E141K, Q142R, S150G, L151P, V160A, T192A, M209V, and I305V amino acid substitutions,
rr) Q65P, 187V, R118S, E141K, Q142R, S150G, L151P, T192A, and I305V amino acid substitutions,
ss) Q65P, 187V, R118S, E141K, S150G, L151P, T192A, M209V, and I305V amino acid substitutions,
tt) Q65P, R118S, S150G, Q142R, L151P, T192A, M209V, and I305V amino acid substitutions,
uu) Q65P, 187V, R118S, E141K, S150G, L151P, V160A, T192A, M209V, and I305V amino acid substitutions,
vv) Q65P, 187V, R118S, E141K, Q142R, S150G, L151P, V160A, T192A, I201V, M209V, and I305V amino acid substitutions,
ww) Q65P, 187V, R118S, K140R, E141K, Q142R, S150G, L151P, V160A, T192A, M209V, K267R, I305V, and E313K amino acid substitutions,
xx) Q65P, I87V, R118S, E141K, Q142R, S150G, L151P, V160A, T192A, M209V, I305V, I306V, and E313K amino acid substitutions,
yy) Q65P, I87V, 198V, R118S, E141K, Q142R, S150G, L151P, V160A, T192A, M209V, and I305V amino acid substitutions,
zz) Q65P, I87V, R118S, E141K, Q142R, S150G, L151P, V160A, T192A, A199T, M209V, D303G, and I305V amino acid substitutions,
aaa) Q65P, 187V, R118S, G120R, E141K, Q142R, S150G, L151P, V160A, T192A, D197G, M209V, M241V, I305V, and E313K amino acid substitutions,
bbb) Q65P, I87V, R118S, E141K, Q142R, S150G, L151P, V160A, V162A, A166V, T192A, M209V, I305V, and E313K amino acid substitutions,
ccc) Q65P, 187V, R118S, E141K, Q142R, S150G, L151P, V160A, T192A, M209V, I305V, E307R, and E313K amino acid substitutions,
ddd) E27D, Q65P, 187V, R118S, E141K, Q142R, S150G, L151P, V160A, T192A, M209V, E251K, and I305V amino acid substitutions,
eee) Q65P, 187V, R118S, E141K, Q142R, S150G, L151P, V160A, T192A, M209V, M241T, and I305V amino acid substitutions,
fff) Q65P, I87V, R118S, E141K, Q142R, S150G, L151P, V160A, T192A, M209V, N270D, and I305V amino acid substitutions,
ggg) K2R, F51L, Q65P, K70T, 187V, R118S, E141K, Q142R, S150G, L151P, V160A, T192A, M209V, I305V, 1306V, and E313K amino acid substitutions,
hhh) D (1-63), Q65P, K70T, 187V, R118S, E141K, Q142R, S150G, L151P, V160A, T192A, M209V, I305V, I306V, and E313K amino acid substitutions,
iii) D (1-63), Q65P, I87V, R118S, E141K, Q142R, S150G, L151P, V160A, T192A, M209V, and I305V amino acid substitutions,
jjj) Q65P, 187V, R118S, E141K, Q142R, S150G, L151P, V160A, T192A, M209V, L252R, I305V, K307R, and G311D amino acid substitutions,
kkk) Q65P, 187V, R118S, E141K, Q142R, S150G, L151P, V160A, K168E, T192A, M209V, K267E, and I305V amino acid substitutions,
lll) Q65P, 187V, R118S, E141K, Q142R, S150G, L151P, V160A, T192A, M209V, E251K, and I305V amino acid substitutions,
mmm) D(1-63), Q65P, I87V, R118S, E141K, Q142R, S150G, L151P, V160A, T192A, M209V, E251K, and I305V amino acid substitutions, nnn) Q65P, 187V, R118S, E141K, Q142R, S150G, L151P, V160A, T192A, M209V, M241T, and I305V amino acid substitutions,
ooo) D(1-63), Q65P, 187V, R118S, E141K, Q142R, S150G, L151P, V160A, T192A, M209V, M241T, and I305V amino acid substitutions,
ppp) D(1-63) Q65P, I87V, R118S, E141K, Q142R, S150G, L151P, V160A, T192A, M209V, M241T, I305V amino acid substitutions,
qqq) D(1-63), Q65P, 187V, R118S, E141K, Q142R, S150G, L151P, V160A, T192A, M209V, and I305V amino acid substitutions,
rrr) D(1-63), Q65P, I87V, R118S, E141K, Q142R, S150G, L151P, V160A, L179M, T192A, M209V, N232S, I305V, and I306T amino acid substitutions,
sss) D(1-63), Q65P, I87V, R118S, E141K, Q142R, S150G, L151P, V160A, T192A, M209V, I285V, and I305V amino acid substitutions,
ttt) D(1-63), Q65P, I87V, R118S, E141K, Q142R, S150G, L151P, V160A, T192A, M209V, S289G, I305V, and M310V amino acid substitutions,
uuu) D(1-63), Q65P, 187V, R118S, E141K, Q142R, S150G, L151P, V160A, D167G, T192A, M209V, N232S, and I305V amino acid substitutions,
vvv) D(1-63), Q65P, 187V, 199V, R118S, E141K, Q142R, S150G, L151P, V160A, T192A, M209V, I305V, K307Q, and M310T amino acid substitutions,
www) Q65P, 187V, R118S, E141K, Q142R, S150G, L151P, V160A, T192A, M209V, and I305V amino acid substitutions,
xxx) Q65P, 187V, S89N, R118S, E141K, Q142R, S150G, L151P, V160A, T192A, M209V, and I305V amino acid substitutions,
yyy) D(1-63) Q65P, I87V, R118S, E141K, Q142R, S150G, L151P, V160A, D167G, T192A, M209V, M241T, N232S, and I305V amino acid substitutions,
zzz) Q65P, 187V, S89N, R118S, E141K, Q142R, S150G, L151P, V160A, T192A, M209V, M241T, and I305V amino acid substitutions,
aaaa) D(1-63), Q65P, I87V, D88G, R118S, E141K, Q142R, S150G, L151P, V160A, T192A, M209V, K283E, and I305V amino acid substitutions,
bbbb) D(1-63), Q65P, 187V, Y111H, R118S, E141K, Q142R, S150G, L151P, V160A, T192A, M209V, and I305V amino acid substitutions,
cccc) D(1-63), Q65P, I87V, R118S, E141K, Q142R, S150G, L151P, V160A, T192A, M209V, S263P, and I305V amino acid substitutions,
dddd) D(1-63), Q65P, I87V, R118S, E141K, Q142R, S150G, L151P, V160A, T192A, K194I, M209V, and I305V amino acid substitutions,
eeee) D(1-63), Q65P, 187V, R118S, E141K, Q142R, S150G, L151P, V160A, T192A, M209V, and I305V amino acid substitutions,
ffff) D(1-63), Q65P, I87V, R118S, E141K, Q142R, S150G, L151P, V160A, T192A, M209V, M241T, and I305V amino acid substitutions,
gggg) D(1-63), Q65P, I87V, R118S, E141K, Q142R, S150G, L151P, V160A, T192A, K194I, M209V, and I305V amino acid substitutions,
hhhh) D(1-63), Q65P, I87V, R118S, E141K, Q142R, S150G, L151P, V160A, T192A, M209V, M241T, S263P, and I305V amino acid substitutions,
iiii) D(1-63), Q65P, I87V, R118S, E141K, Q142R, S150G, L151P, V160A, T192A, K194I, M209V, M241T, and I305V amino acid substitutions,
jjjj) D(1-63), Q65P, I87V, R118S, E141K, Q142R, S150G, L151P, V160A, T192A, K194I, M209V, S263P, and I305V amino acid substitutions,
kkkk) D(1-63), Q65P, I87V, R118S, E141K, Q142R, S150G, L151 P, V160A, T192A, M209V, and I305V amino acid substitutions,
llll) D(1-63), Q65P, 187V, R118S, E141K, Q142R, S150G, L151 P, V160A, T192A, K194I, M209V, M241T, and I305V amino acid substitutions,
mmmm) D(1-63), Q65P, I87V, R118S, E141K, Q142R, S150G, L151 P, V160A, T192A, K1941, M209V, M241T, S263P, and I305V amino acid substitutions,
nnnn) D(1-63), Q65P, I87V, R118S, E141K, Q142R, S150G, L151P, V160A, T192A, K194I, M209V, S263P, and I305V amino acid substitutions,
oooo) Q65P, I87V, R118S, E141K, Q142R, S150G, L151P, V160A, T192A, M209V, S263P, and I305V amino acid substitutions,
pppp) Q65P, I87V, R118S, E141K, Q142R, S150G, L151P, V160A, T192A, K194I, M209V, and I305V amino acid substitutions,
qqqq) Q65P, I87V, R118S, E141K, Q142R, S150G, L151P, V160A, T192A, M209V, M241T, S263P, and I305V amino acid substitutions,
rrrr) Q65P, I87V, R118S, E141K, Q142R, S150G, L151P, V160A, T192A, K194I, M209V, M241T, and I305V amino acid substitutions, and
ssss) Q65P, I87V, R118S, E141K, Q142R, S150G, L151P, V160A, T192A, K194I, M209V, M241T, S263P, and I305V amino acid substitutions.

The biotin ligase in aspects of this invention may comprise:
a) a polypeptide comprising an amino acid sequence selected from the group consisting of the sequence A, the sequence of which is:

```
Ile Pro Leu Leu Asn Ala Lys Gln Ile Leu Gly Gln Leu Asp Gly Gly
1 5 10 15
Ser Val Ala Val Leu Pro Val Val Asp Ser Thr Asn Gln Tyr Leu Leu
                        20 25 30
Asp Arg Ile Gly Glu Leu Lys Ser Gly Asp Ala Cys Ile Ala Glu Tyr
                35 40 45
Gln Gln Ala Gly Arg Gly Ser Arg Gly Arg Lys Trp Phe Ser Pro Phe
        50 55 60
Gly Ala Asn Leu Tyr Leu Ser Met Phe Trp Arg Leu Lys Arg Gly Pro
65 70 75 80
Ala Ala Ile Gly Leu Gly Pro Val Ile Gly Ile Val Met Ala Glu Ala
                                85 90 95
Leu Arg Lys Leu Gly Ala Asp Lys Val Arg Val Lys Trp Pro Asn Asp
                        100 105 110
Leu Tyr Leu Gln Asp Arg Lys Leu Ala Gly Ile Leu Val Glu Leu Ala
                115 120 125
Gly Ile Thr Gly Asp Ala Ala Gln Ile Val Ile Gly Ala Gly Ile Asn
        130 135 140
Val Ala Met Arg Arg Val Glu Glu Ser Val Val Asn Gln Gly Trp Ile
145 150 155 160
Thr Leu Gln Glu Ala Gly Ile Asn Leu Asp Arg Asn Thr Leu Ala Ala
                                165 170 175
Met Leu Ile Arg Glu Leu Arg Ala Ala Leu Glu Leu Phe Glu Gln Glu
                        180 185 190
Gly Leu Ala Pro Tyr Leu Ser Arg Trp Glu Lys Leu Asp Asn Phe Ile
                195 200 205
Asn Arg Pro Val Lys Leu Ile Ile Gly Asp Lys Glu Ile Phe Gly Ile
        210 215 220
Ser Arg Gly Ile Asp Lys Gln Gly Ala Leu Leu Leu Glu Gln Asp Gly
225 230 235 240
Val Ile Lys Pro Trp Met Gly Gly Glu Ile Ser Leu Arg Ser Ala Glu
                                245 250 255
Lys
```

and the sequence B, the sequence of which is:

```
Lys Asp Asn Thr Val Pro Leu Lys Leu Ile Ala Leu Leu Ala Asn Gly
1 5 10 15
Glu Phe His Ser Gly Glu Gln Leu Gly Glu Thr Leu Gly Met Ser Arg
                        20 25 30
Ala Ala Ile Asn Lys His Ile Gln Thr Leu Arg Asp Trp Gly Val Asp
                35 40 45
Val Phe Thr Val Pro Gly Lys Gly Tyr Ser Leu Pro Glu Pro Ile Pro
        50 55 60
Leu Leu Asn Ala Lys Gln Ile Leu Gly Gln Leu Asp Gly Gly Ser Val
65 70 75 80
Ala Val Leu Pro Val Val Asp Ser Thr Asn Gln Tyr Leu Leu Asp Arg
                                85 90 95
Ile Gly Glu Leu Lys Ser Gly Asp Ala Cys Ile Ala Glu Tyr Gln Gln
                        100 105 110
Ala Gly Arg Gly Ser Arg Gly Arg Lys Trp Phe Ser Pro Phe Gly Ala
                115 120 125
Asn Leu Tyr Leu Ser Met Phe Trp Arg Leu Lys Arg Gly Pro Ala Ala
        130 135 140
Ile Gly Leu Gly Pro Val Ile Gly Ile Val Met Ala Glu Ala Leu Arg
145 150 155 160
Lys Leu Gly Ala Asp Lys Val Arg Val Lys Trp Pro Asn Asp Leu Tyr
                                165 170 175
Leu Gln Asp Arg Lys Leu Ala Gly Ile Leu Val Glu Leu Ala Gly Ile
                        180 185 190
Thr Gly Asp Ala Ala Gln Ile Val Ile Gly Ala Gly Ile Asn Val Ala
                195 200 205
Met Arg Arg Val Glu Glu Ser Val Val Asn Gln Gly Trp Ile Thr Leu
        210 215 220
Gln Glu Ala Gly Ile Asn Leu Asp Arg Asn Thr Leu Ala Ala Thr Leu
225 230 235 240
Ile Arg Glu Leu Arg Ala Ala Leu Glu Leu Phe Glu Gln Glu Gly Leu
                                245 250 255
Ala Pro Tyr Leu Pro Arg Trp Glu Lys Leu Asp Asn Phe Ile Asn Arg
                        260 265 270
Pro Val Lys Leu Ile Ile Gly Asp Lys Glu Ile Phe Gly Ile Ser Arg
                275 280 285
Gly Ile Asp Lys Gln Gly Ala Leu Leu Leu Glu Gln Asp Gly Val Ile
        290 295 300
Lys Pro Trp Met Gly Gly Glu Ile Ser Leu Arg Ser Ala Glu Lys,
305 310 315
```

and a polypeptide comprising an amino acid sequence having at least 90% sequence identity to sequence A or sequence B and wherein said polypeptide is a biotin ligase for proximity-dependent biotinylation of proteins.

The antibody in compositions and conjugates of this invention serves as targeting sequence that directs the biotin ligase to a subcellular region, or a cellular component or an epitope of interest.

The targeting antibody is preferably selected from the group consisting of antibodies that bind to a secretory protein, a membrane protein, a nuclear protein, a mitochondrial protein, an outer mitochondrial membrane protein, an endoplasmic reticulum protein, an endoplasmic reticulum membrane protein, a nucleolar protein, a nuclear export protein, a peroxisome protein.

The protein of interest for biotinylation may be a cytosolic protein, a nuclear protein, a membrane protein, a mitochondrial protein, a P-body protein, a secretory pathway protein, or an antibody specific for an epitope in the subcellular region of interest.

The protein of interest may also be a protein that is for instance ectopically expressed, or of which the expression is the result of genetic modification, such as a protein fusion, e.g. as generated using genetic knock-in strategies. Such proteins may all be targets for a method for biotinylating a protein as described herein, and one of skill in the art is familiar with the methods for producing a targeting antibody to such targets.

Antibodies capable of binding chemical or post-translational modifications on proteins or nucleic acids including DNA and RNA may also be targeted by ProtA-Turbo. Furthermore, antibodies targeting metabolites, fatty acids or sugars may also be used in aspects of this invention.

In a method of the present invention as described herein for biotinylating a protein, the method may further comprise the step of isolating the biotinylated proteins using a biotin-binding protein that binds to the biotinylated proteins, preferably such a biotin-binding protein is streptavidin or avidin.

In a method of the present invention as described herein for biotinylating a protein, the method may further comprise the step of labeling the biotinylated proteins with a biotin-binding protein conjugated to a detectable label, preferably such a detectable label is fluorescent, bioluminescent, or chemiluminescent.

In a method of the present invention as described herein for biotinylating a protein, the method may further comprise the step of imaging luminescence emitted from the detectable label.

In a method of the present invention as described herein for biotinylating a protein, the method may further comprise the step of identifying at least one biotinylated protein.

In a method of the present invention as described herein for biotinylating a protein, said identifying may comprise performing mass spectrometry, liquid chromatography-mass spectrometry (LC/MS), an enzyme-linked immunosorbent assay (ELISA), a Western blot, immunostaining, high-performance liquid chromatography (HPLC), protein sequencing, or peptide mass fingerprinting.

The fusion polypeptide comprising the biotin ligase may be provided by or may be comprised in a vector comprising a promoter operably linked to a polynucleotide encoding the fusion polypeptide. The fusion polypeptide of the invention may for instance be produced by recombinant expression of any expression system suitable for production of protein fusions, comprising the sequences of the biotin ligase and the immunoglobulin-binding bacterial protein.

In the fusion polypeptide, the immunoglobulin-binding bacterial protein may be fused to the biotin ligase via a linker, such as a peptide linker. A preferred linker group is a linker polypeptide comprising from 1 to about 60 amino acid residues, preferably from 5 to about 40 amino acid residues, most preferred about 15 amino acid residues such as 10 amino acid residues, 11 amino acid residues, 12 amino acid residues, 13 amino acid residues, 14 amino acid residues, 15 amino acid residues, 16 amino acid residues, 17 amino acid residues, 18 amino acid residues, 19 amino acid residues or 20 amino acid residues. Some preferred examples of such amino acid sequences include Gly-Ser linkers, for example of the type (Glyx Sery)z such as, for example, (Gly4 Ser)3, (Gly4 Ser)7 or (Gly3 Ser2)3, as described in WO 99/42077, and the GS30, GS15, GS9 and GS7 linkers described in, for example, WO 06/040153 and WO 06/122825, as well as hinge-like regions, such as the hinge regions of naturally occurring heavy chain antibodies or similar sequences (such as described in WO 94/04678). A most preferred linker is a (Gly4Ser)3 linker.

In preferred embodiments of this invention, the biotin derivative may be desthiobiotin.

In a method of the present invention, the cell of interest (the target cell), may be permeabilized prior to contacting the cell with the complex of the present invention.

In embodiments described herein, the fusion polypeptide comprising the immunoglobulin-binding bacterial protein and biotin ligase enzyme may be contacted with the permeabilized cells separate, subsequent or simultaneously with the antibody, or the fusion polypeptide and antibody may be complexed and contacted with the permeabilized cells in the form of a complex.

The antibody in aspects of this invention is an antibody to which the immunoglobulin-binding bacterial protein in the fusion polypeptide of the invention can bind.

The antibody in aspects of this invention may target the fusion polypeptide to proteins or post-translational modifications of interest. Addition of biotin then triggers bait-proximal protein biotinylation. Biotinylated proteins can subsequently be enriched from crude lysates and identified by mass spectrometry.

The targeting immunoglobulin in aspects of this invention specifically interacts with a protein of interest that is to be biotinylated.

Embodiments described herein may be combined.

Immunoglobulin G-binding protein A from
Staphylococcus aureus (UniProtKB - P38507)
MKKKNIYSIRKLGVGIASVTLGTLLISGGVTPAANAAQHDEAQQNAFYQV
LNMPNLNADQRNGFIQSLKDDPSQSANVLGEAQKLNDSQAPKADAQQNKE
NKDQQSAFYEILNMPNLNEEQRNGFIQSLKDDPSQSTNVLGEAKKLNESQ
APKADNNENKEQQNAFYEILNMPNLNEEQRNGFIQSLKDDPSQSANLLAE
AKKLNESQAPKADNKFNKEQQNAFYEILHLPNLNEEQRNGFIQSLKDDPS
QSANLLAEAKKLNDAQAPKADNKFNKEQQNAFYEILHLPNLTEEQRNGFI
QSLKDDPSVSKEILAEAKKLNDAQAPKEEDNNKPGKEDGNKPGKEDGNKP
GKEDNKKPGKEDGNKPGKEDNKKPGKEDGNKPGKEDGNKPGKEDGNKPGK
EDGNKPGKEDGNGVHVVKPGDTVNDIAKANGTTADKIAADNKLADKNMIK
PGQELVVDKKQPANHADANKAQALPETGEENPFIGTTVFGGLSLALGAAL
LAGRRREL
Immunoglobulin G-binding protein A from
Staphylococcus aureus (UniProtKB - P38507)
after processing (1-36 and 478-508 are
removed) the following sequence remains
AQHDEAQQNAFYQVLNMPNLNADQRNGFIQSLKDDPSQSANVLGEAQKLN
DSQAPKADAQQNKFNKDQQSAFYEILNMPNLNEEQRNGFIQSLKDDPSQS
TNVLGEAKKLNESQAPKADNNFNKEQQNAFYEILNMPNLNEEQRNGFIQS
LKDDPSQSANLLAEAKKLNESQAPKADNKENKEQQNAFYEILHLPNLNEE
QRNGFIQSLKDDPSQSANLLAEAKKLNDAQAPKADNKFNKEQQNAFYEIL
HLPNLTEEQRNGFIQSLKDDPSVSKEILAEAKKLNDAQAPKEEDNNKPGK
EDGNKPGKEDGNKPGKEDNKKPGKEDGNKPGKEDNKKPGKEDGNKPGKED
GNKPGKEDGNKPGKEDGNKPGKEDGNGVHVVKPGDTVNDIAKANGTTADK
IAADNKLADKNMIKPGQELVVDKKQPANHADANKAQALPET
Immunoglobulin G-binding protein G from
Streptococcus sp. group G (UniProtKB -
P19909)
MEKEKKVKYFLRKSAFGLASVSAAFLVGSTVFAVDSPIEDTPIIRNGGEL
TNLLGNSETTLALRNEESATADLTAAAVADTVAAAAAENAGAAAWEAAAA
ADALAKAKADALKEFNKYGVSDYYKNLINNAKTVEGVKDLQAQVVESAKK
ARISEATDGLSDFLKSQTPAEDTVKSIELAEAKVLANRELDKYGVSDYHK
NLINNAKTVEGVKDLQAQVVESAKKARISEATDGLSDFLKSQTPAEDTVK
SIELAEAKVLANRELDKYGVSDYYKNLINNAKTVEGVKALIDEILAALPK
TDTYKLILNGKTLKGETTTEAVDAATAEKVFKQYANDNGVDGEWTYDDAT
KTFTVTEKPEVIDASELTPAVTTYKLVINGKTLKGETTTEAVDAATAEKV
FKQYANDNGVDGEWTYDDATKTFTVTEKPEVIDASELTPAVTTYKLVING
KTLKGETTTKAVDAETAEKAFKQYANDNGVDGVWTYDDATKTFTVTEMVT
EVPGDAPTEPEKPEASIPLVPLTPATPIAKDDAKKDDTKKEDAKKPEAKK
EDAKKAETLPTTGEGSNPFFTAAALAVMAGAGALAVASKRKED
Immunoglobulin G-binding protein G from
Streptococcus sp. group G (UniProtKB -
P19909) after processing (1-33 and
563-593 are removed)
VDSPIEDTPIIRNGGELTNLLGNSETTLALRNEESATADLTAAAVADTVA
AAAAENAGAAAWEAAAAADALAKAKADALKEFNKYGVSDYYKNLINNAKT
VEGVKDLQAQVVESAKKARISEATDGLSDFLKSQTPAEDTVKSIELAEAK
VLANRELDKYGVSDYHKNLINNAKTVEGVKDLQAQVVESAKKARISEATD
GLSDFLKSQTPAEDTVKSIELAEAKVLANRELDKYGVSDYYKNLINNAKT
VEGVKALIDEILAALPKTDTYKLILNGKTLKGETTTEAVDAATAEKVFKQ
YANDNGVDGEWTYDDATKTFTVTEKPEVIDASELTPAVTTYKLVINGKTL
KGETTTEAVDAATAEKVFKQYANDNGVDGEWTYDDATKTFTVTEKPEVID
ASELTPAVTTYKLVINGKTLKGETTTKAVDAETAEKAFKQYANDNGVDGV
WTYDDATKTFTVTEMVTEVPGDAPTEPEKPEASIPLVPLTPATPIAKDDA
KKDDTKKEDAKKPEAKKEDAKKAETLPTT

### EXAMPLES

### Example 1

### Materials and Methods

### Cell Culture

HeLa and MCF7 cells were cultured in Dulbecco's modified Eagle medium (DMEM, Gibco) supplemented with 10% FBS and penicillin/streptomycin. U397 cells were cultured in RPMI with 10% FBS and penicillin/streptomycin. Low passage primary fibroblasts from Coriell institute (AG08469) were cultured with DMEM supplemented with 15% FBS and penicillin/streptomycin, and used in assays at maximum passage 12.

### Primers and Oligonucleotides

A full list of primers and oligonucleotides used in this work are provided in Table 1.

### Recombinant Protein Purification

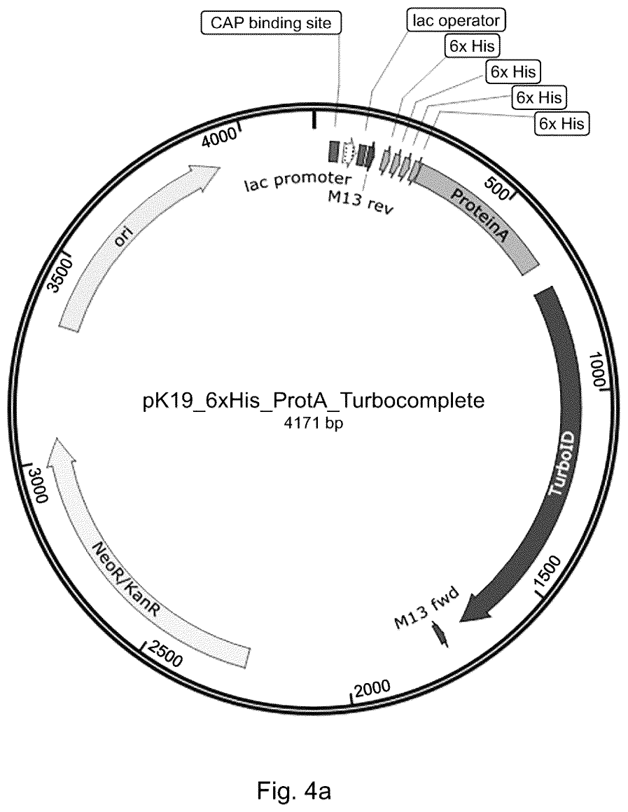

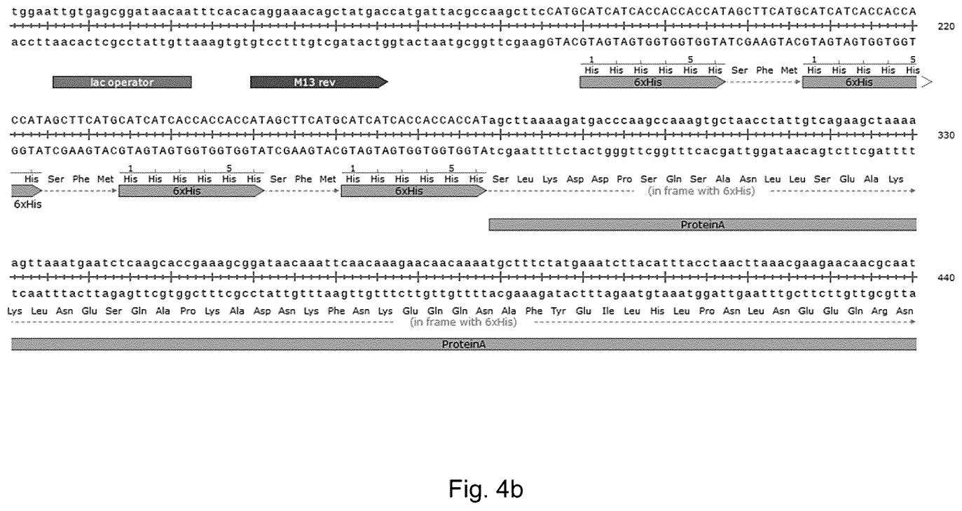

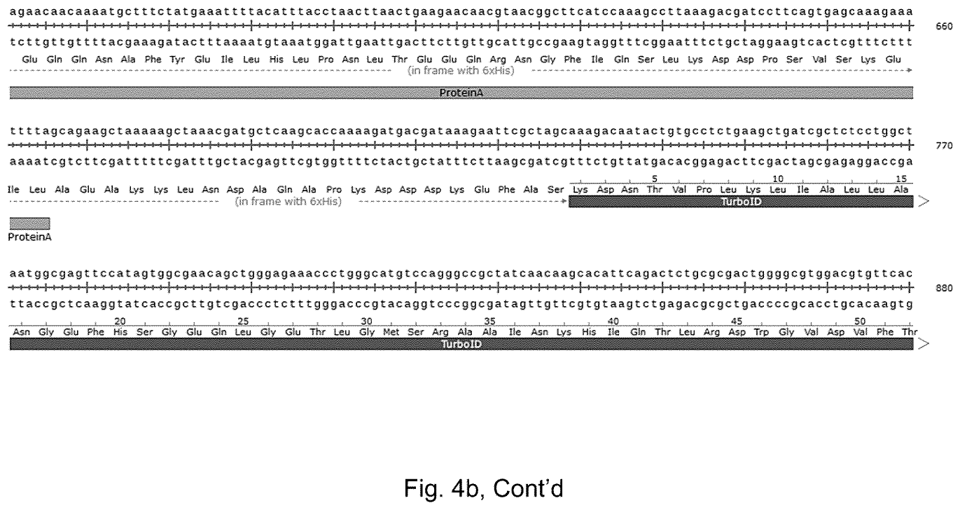

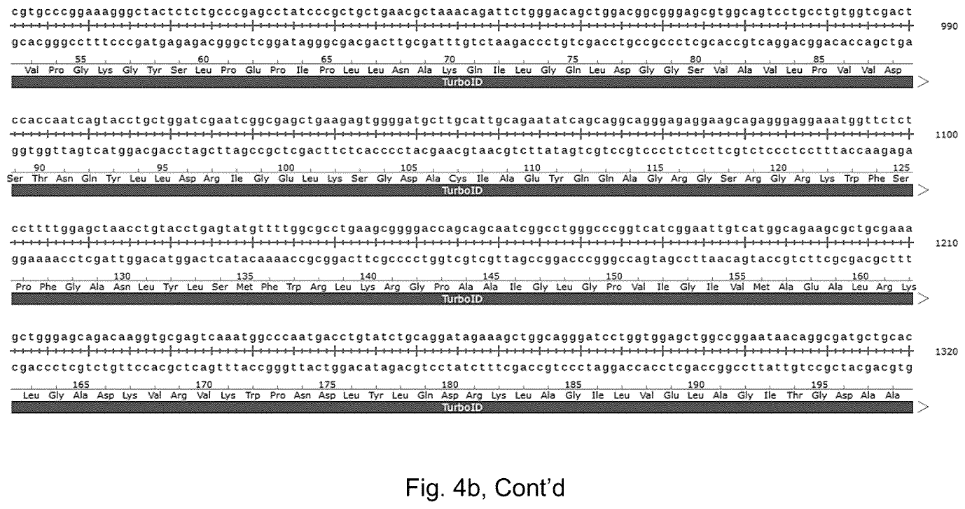

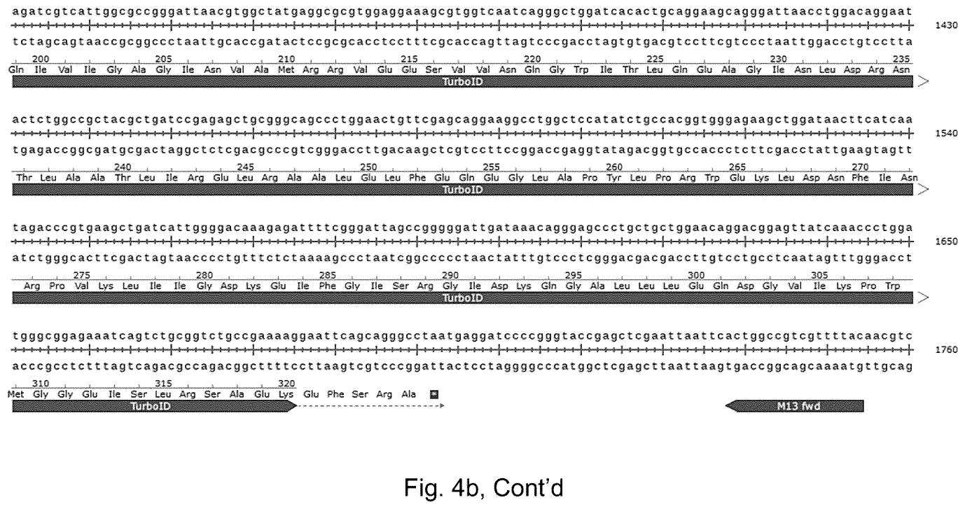

Mnase sequence was substituted by TurboID sequence in the pK19pAMNase vector (Addgene #86973; FIG. 4). TurboID was amplified from the plasmid 3xHA TurboID NLS pcDNA3 (Addgene #107171) and inserted in the pK19 vector between EcoRI and BamHI restriction enzymes using the primers TurboEcoRI-F, TurboBamHIfrag-R, TurboBamHIfrag-F and TurboBamHI-R. A histidine tail was added later in the N-terminal part of the sequence of the protein using the oligos 6xHistagHinDIII-F and 6xHistagHinDIII-R in order to facilitate the purification of the enzyme.

The pK19 Protein A Turbo plasmid was transformed in C3013 E.coli (New England Biolabs). Different colonies were grown overnight and cultured in 2L LB medium until reaching OD 0.6. The protein expression was induced for 3 hours with 2 mM IPTG. The bacteria were pelleted and lysed with the following buffer (0.5M NaCl, 10% Glycerol, 20 mM Hepes pH=8, 1 mM EDTA pH=8, 0.1% NP40, 20 mM b-mercaptoethanol and 1 mM PMSF) and sonicated. The lysate was centrifuged for one hour at 12,000×g at 4° C. to eliminate debris. The protein extract was drained by gravity flow in a column with nickel beads, then, the column was washed once with lysis buffer and twice with wash buffer (10 mM Tris, 0.5 mM EDTA, 10% Glycerol, 500 mM NaCl). A pre-elution was performed with 5 ml of 15 mM Imidazol diluted in the wash buffer and 1 ml fractions were collected. The elution of the ProtA-Turbo was mainly obtained with 10 ml of 100 mM Imidazol and also collected in 1 ml fractions. Pre-elutions and elutions were collected in different fractions to analyse the amount and purity of the protein. The collected fractions were run on an SDS-PAGE gel and stained with Imperial Protein Staining (Thermo Scientific) following the manufacturer's instructions. Fractions were pooled according to their similarity in gel and were snap frozen and stored at −80° C. until their use.

### ProtA-Turbo Immunofluorescence

Cells were cultured in coverslips (15 mm diameter) in a 12 well plate. Following day, they were fixed with 4% PFA in PBS for 15 minutes, washed 3 times with PBS, permeabilized in 0.3% Triton in PBS for 10 minutes and blocked in blocking solution (3% BSA in 0.3% Triton-PBS) for 30 minutes. Coverslips were incubated for one hour in a humid chamber with primary antibody diluted 1:150 in blocking solution (H3K9me3 antibody (Abcam, ab8898), BRG1 antibody (Bethyl, A300-813A) and Emerin antibody (10351-1-AP, Proteintech)). Coverslips were washed 4 times with PBS and incubated with 0.2 μl ProtA-Turbo diluted in 30 μl of blocking buffer per coverslip for one hour and washed 4 times with PBS. Then, they were incubated with the biotin reaction buffer (5 mM MgC12, 5 μM Biotin, 1 mM ATP in PBS) during 10 minutes at 37C and washed. Finally, they were incubated with secondary antibody (anti rabbit Alexa fluor 568), FITC-Avidin and DAPI during one hour, washed 4 times with PBS and mounted with fluoromount (Thermo Fisher Scientific). Images were acquired with a confocal microscope LSM900 (ZEISS) and analyzed with Fiji software.

### Prot A-Turbo Targeting in Fixed Cells

Cells were fixed with 4% PFA during 15 minutes, then scraped and collected. Around at least 70 μl of pellet was used for each antibody. Then, cell pellet was incubated on ice with 1ml of Hypotonic Lysis Buffer (10 mM Tris pH 7.5, 10 mM NaCl, 3 mM MgC12, 0.3% NP40 and 10% glycerol) for 10 minutes, centrifuged at 800×g for 8 minutes at 4° C. in order to isolate the nuclei. Nuclei were washed with the same buffer three times and centrifuged at 200×g for 2 minutes at 4° C. Nuclei were washed once with PBS1X. From this step, the centrifugations were performed at room temperature at 1000-3000 rpm for 3 minutes. Nuclei were permeabilized for 10 minutes in rotation with 1 ml of 0.3% Triton-PBS1X and, then, they were blocked with 1 ml of BSA in 0.3% PBS Triton for 30 minutes in rotation. Nuclei were incubated with 3 μg of primary antibody diluted in 300 μl blocking solution (IgG rabbit antibody (12-370, EMD Millipore), BRG1 antibody (Bethyl, A300-813A) and emerin antibody (10351-1-AP, Proteintech)) in rotation for one hour. In order to eliminate the unbound antibody, nuclei were washed twice with PBS1X. Then, nuclei were incubated with 5 μl of protA Turbo diluted in 300 μl blocking solution in rotation for one hour. The unbound protA-turbo fraction was eliminated washing twice the pellet with PBS1X. Then, the nuclei were incubated in a shaker at 37° C. for 10 minutes with 300 μl of biotin reaction buffer (5 mM MgC12, 5 μM Biotin, 1 mM ATP in PBS1X). Nuclei pellet was washed once with PBS1X and lysed with 300 μl Ripa buffer (50 Mm Tris pH7.8, 150 mM NaCl, 0.5% Sodium deoxycholate, 0.1% SDS, 1% NP40) overnight at 4° C. on ice.

The following day, samples were sonicated with a Bioruptor sonicator and decrosslinked with 50 μl of 10% SDS at 95° C. for one hour. An additional cycle with the sonicator was performed and they were centrifuged at maximum speed 10 minutes at 4° C. Then, supernatant was recovered and incubated with 25 μl slurry Streptavidin Sepharose High Performance beads (15511301, Cytiva) for two hours in rotation. Agarose beads were washed 5 times with Ripa buffer. At this point of the protocol, if the purpose was to perform a Western Blot, protein loading buffer (125 mM Tris pH6.8, 25% glycerol, 5% SDS, 0.1% bromophenol blue, 1.43M b-mercatoethanol) were added to the beads, then, the beads were boiled at 95° C. for 10 minutes and then samples were loaded in a SDS-Page gel in order to continue with the Western Blot protocol. However, if the purpose was to analyze the sample by mass spectrometry, after the washes with Ripa buffer, the beads were washed 4 times with PBS1X buffer. In order to elute the proteins, 50 μl elution buffer (2M Urea, 10 mM DTT, 100 mM Tris pH8) was added to the beads and it was incubated during 20 minutes in a shaker. Then, iodoacetamide was added to the samples to a final concentration of 50 mM and samples were incubated in a shaker in dark for 10 minutes. 2.5 μl of trypsin (0.1 mg/ml trypsin stock solution) was added to the sample and incubated in a shaker for 2 hours. Samples were centrifuged and the elutions were saved. Additional 50 μl of elution buffer was added to the beads, incubated for 5 minutes and then, beads were centrifuged and elutions were combined. Peptides were incubated with an additional 1 μl of trypsin overnight. Following day, peptides were acidified with 10 μl of TFA 10% and cleaned using C18 Stagetips²¹. Peptides in Stagetips were stored at 4° C. until the day they were measured by mass spectrometry.

When H3K9me3 (Abcam, ab8898) was used, nuclei isolation was performed before the fixation. Briefly, cells were washed with PBS1X, scraped and transferred to an Eppendorf. Then, cell pellet was incubated on ice with 1 ml of Hypotonic Lysis Buffer (10 mM Tris pH 7.5, 10 mM NaCl, 3 mM MgC12, 0.3% NP40 and 10% glycerol) for 10 minutes, then centrifuged at 800×g for 8 minutes at 4° C. Nuclei were washed with the same buffer three times and centrifuged at 200×g for 2 minutes at 4° C. Nuclei were washed once with PBS1X. Then, nuclei were fixed using 1 ml of 4% PFA and they were incubated in rotation for 15 minutes. After that, nuclei were washed twice with PBS1X and permeabilized in order to continue with the protocol.

### ProtA-Turbo Targeting in Unfixed Cells

Cells were washed with PBS1x and scraped. Around 40 μl of pellet was used for each antibody. Cell pellets were incubated with digitonin buffer (0.04% digitonin diluted in 20 mM HEPES pH7.5, 150 mM NaCl, 0.5 mM Spermidine) for 10 minutes in rotation. After that, 7 μl of sample was analyzed with trypan blue in Countess Cell Counter (Invitrogen), if there was at least 80% non-viable (ensuring sufficient outer membrane permeabilization), samples were treated for the next step, otherwise more digitonin buffer or a higher digitonin concentration was added to reach >80% non-viable cells. Then, cells were centrifuged at room temperature for 3 minutes at 1000-3000 rpm and 2 μg of antibody (Emerin, BRG1 and H3K9me3) diluted in 200 μl digitonin buffer was added to the samples. Sample and antibody were incubated at room temperature for 20 minutes in a shaker at 600 rpm. Afterwards, cells were washed twice with digitonin buffer and they were incubated with 2 μl of ProtA-Turbo diluted in 500 μl digitonin buffer for 30 minutes in rotation at 4° C. Then, cells were washed twice with digitonin buffer. The nuclei were incubated in a shaker at 37° C. during 10 minutes with biotin reaction buffer (5 mM MgC12, 5 μM Biotin, 1 mM ATP in digitonin buffer). Cells were washed once with wash buffer without digitonin, resuspended with 300 μl of RIPA buffer (50 Mm Tris pH7.8, 150 mM NaCl, 0.5% Sodium deoxycholate, 0.1% SDS, 1% NP40) and incubated overnight at 4° C. on ice. Following day, the samples were sonicated in a Bioruptor sonicator until the mix became clear. Then, they were centrifuged at maximum speed at 4° C. for 10 minutes and the supernatant was incubated with streptavidin beads. From this step, the procedure is the same as the one performed in fixed material.

When H3K9me3 (Abcam, ab8898) was used in HeLa cells, nuclei isolation was performed before the incubation with the antibody. Briefly, cells were washed with PBS1X, scraped and transferred to an Eppendorf. Then, cell pellet was incubated on ice with 1 ml of Hypotonic Lysis Buffer (10 mM Tris pH 7.5, 10 mM NaCl, 3 mM MgC12, 0.3% NP40 and 10% glycerol) for 10 minutes, then centrifuged at 800×g for 8 minutes at 4° C. Nuclei were washed with the same buffer three times and centrifuged at 200×g for 2 minutes at 4° C. Then, cells were washed once with wash buffer (no digitonin) and incubated with antibody. From this step samples were treated the same as for the other antibodies.

### LC-MS/MS Measurements and Data Analysis

Digested peptides were eluted from the C18 Stagetips with buffer B (0.1% formic acid, 80% acetonitrile) and after speedvac buffer A (0.1% formic acid) was added to a total volume of 12 μl and measured on an Easy-nLC1000 (Thermo) connected online either to a Orbitrap Exploris or to a LTQ-Orbitrap-Fusion or to a LTQ-Orbitrap Q-Exactive HFX mass spectrometer (Thermo). The method used for the LTQ-Orbitrap-Fusion and LTQ-Orbitrap Q-Exactive has been described before^{22,23}. For the Orbitrap Exploris, a gradient of 12-30% acetonitrile in 43 minutes, then the percentage increased up to 60% in 10 minutes and up to 95% in 1 minute. Total data collecting time was 60 minutes. The spray voltage was set to 2200V in positive mode. The expected LC peak width was 15 s. The full scan of the peptides was set to a resolution of 120000 in a scan range of 350-1300 m/z. The normalized AGC target was 300% and the maximum injection time was 20 ms.

Raw files were analysed using standard settings of MaxQuant 1.5.1.0. Options LFQ, iBAQ and match between runs were selected. As a search database, the human fasta database updated in 2017 from Uniprot was used. Perseus 1.5.1.0.15 was used to filter proteins flagged as contaminant, reverse or only identified by site. The triplicates were grouped based on experimental condition and only proteins that were reproducible quantified in one of the triplicates were maintained for downstream analyses. Missing values were imputed using default parameters. Statistically different proteins were identified using a t-test (FDR <0.05) and required additionally at least a 1.5-fold change over control samples.

Downstream data visualization was performed in R. Proteins that are known to localize to H3K9me3 domains, nuclear lamina or SWI/SNF complexes were determined using literature curation. Pericentric heterochromatin binding proteins were obtained from pericentromeric purifications in mouse ESCs²⁴. GO-analyses were performed using Clusterprofiler²⁵. For GO-based comparison of ProtA-Turbo Emerin with other published lamin-targeting strategies, we used data for nuclear lamina microdomain mapping (LAP2B-BioID²⁶), a two-component lamina BioID mapping strategy (2C-BioID²⁷), conventional Lamin A BioID3 and an antibody-HRP based method for biotinylation by antibody recognzation (BAR¹⁵). Enriched proteins were defined according to following criteria: LAP2B-BioID >1.5-FC/ctrl; 2C-BioID >1.5-FC/ctrl (-noAP21967); Lamin A BioID: downloaded the proteins in their Table S1; BAR: all proteins of the LMNA Unbound sample that are >2 fold enriched over control, were not shared with the no antibody control and had at least 2 unique peptides. All GO terms were determined using Clusterprofiler and ten GO terms centered around the nuclear lamina were selected for evaluation of method performance. For the interaction network, proteins were selected that were significantly enriched in either H3K9me3 enrichments (HeLa fixed, HeLa unfixed, MCF7 fixed and U937 unfixed) and had additionally at least a two-fold enrichment over IgG control. Proteins present in at least 3 of these datasets were retained and queried using the stringapp plugin of Cytoscape 3.8.2.²⁸. Disconnected nodes, and nodes that had no interaction as determined using experimental evidence or documented in a database, were removed.

### ChIP-Sequencing

Cells were crosslinked with 1% formaldehyde, then, cells were quenched with 125 mM glycine. Cells were washed with PBS1x, scrapped and pelleted. Cell pellet was resuspended in 5ml buffer B (0.25% TritonX-100, 10 mM EDTA, 0.5 mM EGTA and 20 mM Hepes) per 150 cm dish of cells collected and spin at 1600 rpm at 4 C for 5 minutes. Then, pellet was incubated for 10 minutes at 4° C. in rotation with 30 ml buffer C (150 mM NaCl, 1 mM EDTA, 0.5 mM EGTA and 50 mM Hepes) and centrifuged again at 1600 rpm at 4° C. for 5 minutes. Pellet was isolated nuclei. Nuclei were resuspended in 1ml of incubation buffer (0.15% SDS, 1% Triton, 150 mM NaCl, 1 mM EDTA, 0.5 mM EGTA and 20 mM Hepes) and sonicated in a Bioruptor sonicator. Chromatin fragments size was checked by decrosslinking 5 μl of samples and running them in an agarose gel. Fragments around 300 bp were used. 300 μl of chromatin was incubated overnight at 4° C. with 3 μL V5 antibody (P/N 46-0705, Invitrogen), after that, incubated with magnetic Dynabeads protein A/G magnetic beads (10008D and 10009D, Invitrogen). Then, beads were washed once with twice with Buffer 1 (0.1% SDS, 0.1% NaDOC, 1% TritonX-100, 150 mM NaCl, 1 mM EDTA, 0.5 mM EGTA and 20 mM Hepes), once with Buffer 2 (0.1% SDS, 0.1% NaDOC, 1% TritonX-100, 500 mM NaCl, 1 mM EDTA, 0.5 mM EGTA and 20 mM Hepes), once with buffer 3 (0.5% NaDOC, 0.5% NP40, 250 mM LiCl, 1 mM EDTA, 0.5mMEGTA and 20 mM Hepes) and twice with buffer 4 (1 mM EDTA, 0.5 mM EGTA, 20 mM Hepes). For every wash, the beads were incubated with the washing buffer in rotation at 4° C. for 5 minutes. After washes, chromatin was eluted with 200 μl of elution buffer (1% SDS and 0.5 mM NaHCO3) and incubating 20 minutes in rotation at room temperature. Supernatant was collected (chromatin) and beads were discarded. The eluted chromatin was decrosslinked by adding 8 μl of 5M NaCl and 2 μl of 10 mg/ml Proteinase K and incubating at 65° C. shaking at least for 4 hours. DNA was purified using MiniElute columns (Qiagen). The library for sequencing was prepared with Kapa HyperPrep Kit (Kapa Biosystems) essentially following manufacturer instructions and NEXTflex adapters (Bio Scientific) were used. Samples were analysed on an Agilent 2100 Bioanalyser for purity and sequenced on an Illumina NextSeq500.

### ProtA-Turbo ChIP-Sequencing for H3K9me3

ProtA-Turbo protocol in fixed cells for H3K9me3 antibody was used. After washing the biotinylation reaction buffer, nuclei were resuspended in 1 ml of SDS buffer (50 mM Tris pH8, 0.5% SDS, 100 mM NaCl, 5 mM EDTA) and incubated 10 minutes on ice. Then, they were pelleted and resuspended in 300 μl IP buffer (0.3% SDS, 1.1% Triton, 1.2 mM EDTA, 16.7 mM Tris pH8, 167 mM NaCl) and sonicated in a Bioruptor Sonicator. Samples were precleared with 30 μl Dynabeads protein A and 5 ml BSA 5% during 1 h rotation. After that, samples were incubated with 30 μl Streptavidin magnetic beads M280 (Invitrogen) for 3 hours. Then, beads were washed twice with 2% SDS and 3 times with LiCl buffer (100 mM Tris pH8, 500 mM LiCl, 1% NP40, 1% Sodium deoxycholate). Samples were decrosslinked overnight at 65° C. shaking in 60 μl of 300 mM NaCl. Following day, 15 μl of proteinase K buffer (50 mM Tris pH7.5, 25 mM EDTA, 1.25% SDS) and 1.5 μl proteinase K (10 mg/ml) was added to the sample and incubated 2h at 45° C. shaking. Then, the supernatant was collected and the beads were discarded. DNA was purified using MiniElute columns (Qiagen). Library preparation for sequencing was performed as described for FLYWCH1 ChIP-sequencing.

### ChIP-Sequencing Analysis

ChIP-seq libraries were sequenced paired-end on an Illumina Nextseq 500 sequencer. For biotin chip-seq after ProtA-Turbo targeting of H3K9me3 or IgG, the reads were mapped against the hg38 genome build using bwa with parameters mem-t 32²⁹. Reference H3K9me3 ChIP-seq in wild type HeLa cells was downloaded from GEO (accession number GSE86814³⁰) and processed in parallel. Duplicate reads were identified and filtered using Picard tools version 1.1²⁹ (http://broadinstitute.github.io/picard/). FLYWCH1 and IgG ChIP-seq was processed using the seq2science pipeline (10.5281/zenodo.3921913). Parameters were fastp as trimmer, bwa-mem2 as aligners, minimal map quality of 30. For all files, peaks were called using macs2³¹ with q-value 0.001 for H3K9me3 tracks and q 0.05 for FLYWCH1 and IgG ChIP-seq. Bigwig files were visualized in the Integrative Genomics Viewer. ChIP-seq heatmaps were generated using fluff heatmap³². Motif analysis was performed using Homer³³ using default parameters, genome hg38 and width 200. Kayotype plots were generated in R using karyoploteR³⁴. For intersection with repeat elements, the repeat masker database was downloaded using the UCSC table browser. Reads intersecting the repeat coordinates were obtained using bamtools multicov. Only repeat regions that had more than 1000 reads and were at least 10-fold different between FLYWCH1 and IgG ChIP-seq were retained.

### Immunofluorescence

The immunofluorescence was performed using the same protocol in the immunofluorescence for protein A turbo but the incubation with protein A Turbo and biotinylation reaction were omitted.

### FLYWCH1 Overexpression

EGFP FLYWCH1 was cloned in the pEGFP-C3 vector. FLYWCH1 cDNA was amplified using as a template the vector IRATp970F08101D (Biosource) with the primers FLYWCH1EcorI-Fw and FLYWCH1KpnI-Rv. Both vector and PCR product were cut by EcoRI and KpnI and ligate. HeLa cells were transfected with PEI and harvested 48 hours after transfection or immunofluorescence protocol was performed.

### Knock-in Cell Lines

The pUC57 modified plasmid (a kind gift from Jop Kind lab) and the pU6-(BbsI)-Cbh-Cas9-T2A-mCherry (Addgene #64324) plasmid were used for tagging endogenous FLYWCH1 and emerin. For FLYWCH1, the gRNA (GGGTGCTGAGCGTGGCCTGA) was inserted in the pU6-(BbsI)-Cbh-Cas9-T2A-mCherry and it targets in the 5′ of FLYWCH1 sequence. FLYWCH1 homology arms were inserted in both sites of BSD-P2A-GFP-V5 or BSD-P2A-MiniturboID-V5. The gBlock HA1FLYWCH1 was inserted as an homology arm in the vector and the primers HA2FLYWCH1NotI-Fw and HA2FLYWCH1NotI-Rv were used for the amplification of one of the homology arm from cDNA. The primers TurboNheIfrag-F, TurboNheIfrag-R, TurboNotI-R and MiniTurbogoodNheI-F were used to amplify MiniturboID from the vector 3xHA MiniTurboID NLS pcDNA3 (Addgene #107172) and replace GFP and the gBlock 3xV5 to add V5 and a flexible linker as a tag. For Emerin, the gRNA (CGCCCACGCCCGAGTCCGCC) was also inserted in the same vector as for FLYWCH1. Then, Emerin homology arms were inserted in both sites of BSD-P2A-Turbo. Homology arms were amplified from cDNA using the primers HA1emerinMluI-Fw, HA1emerinNcoI-Rv, HA2emerinHindIII-Fw and HA2emerinAscI-Rv. For both FLYWCH1 and Emerin, the length used for the homology arms was approximately 500 bp upstream and downstream from the start transcription site. The two new vectors were transfected as a ratio 1:1 in HeLa cells and then the tagged cells were selected with blasticidin.

### Protein Extract and Streptavidin Pulldown of the Turbo and Miniturbo Tagged Cell Lines

WT and tagged cells were treated with 50 mM biotin (B20656, Life technologies) for one hour. Then, they were washed with PBS1 x and scrapped. They were pelleted and nuclei were isolated using Nuclear Isolation Buffer (15 mM Tris pH7.5, 15 mM NaCl, 60 mM KCl, 5 mM MgCl2, 1 mM CaCl2, 250 mM Sucrose and 0.03% NP40). Cells were incubated in this buffer for 30 minutes at 4° C. in rotation, then, they were centrifuged at 3200×g for 10 minutes at 4° C. Then, isolated nuclei were resuspended in Ripa buffer and sonicated in a Bioruptor until the extract was almost clear. Then, they were centrifuged at maximum speed for 10 minutes at 4° C. to eliminate debris. Supernatant was collected and protein concentration was measured using Bradford assay. Approximately 4 mg of protein per reaction were incubated with 25 μl Streptavidin Sepharose High Performance beads (15511301, Cytiva) and 2 μl Ethidium bromide for 2 hours in rotation at 4° C. Then, samples were treated using the same procedure as it was used for the protA Turbo protocols for mass spectrometry analysis.

### Western Blotting

Western blotting was performed as described previously in any publication. Briefly, samples were boiled at 95° C. for 5 minutes and loaded in an SDS-page. Proteins were transferred from the gel to a nitrocellulose membrane using a Trans-Blot Turbo Transfer System (Bio-Rad) with the standard settings. Membranes were blocked in 5% milk in 0.1% Tween-PBS for 30 minutes or in 5% BSA in 0.1% Tween-PBS for the membranes that were blotting for the biotin. Then, membranes were incubated overnight at 4C with the antibody diluted in blocking solution with milk, except the antibody against V5 that was incubated with 0.1% Tween-PBS. The antibodies used for this study were anti-B-actin (A1978, SIGMA) diluted 1:5000, anti-BRG1 (Bethyl, A300-813A) diluted 1:1000, anti-histone H3 (tri methyl K9) (a-H3K9me3) (Abcam, ab8898) diluted 1:1000, anti-emerin polyclonal (10351-1-AP, Proteintech) diluted 1:1000 and V5 Tag monoclonal (P/N 46-0705, Invitrogen, now R960-25 ThermoFisher) diluted 1:1000. Then, membranes were incubated with HRP secondary antibody from. Membranes blotted for biotin were incubated with HRP-Streptavidin (5911, Invitrogen) diluted 1:1000 in blocking solution with BSA for 1 hour at room temperature. All of the membranes were developed using Supersignal West Pico Plus Chemiluminescent Substrate reagent (34580, Thermo Scientific) and imaged using ImageQuant LAS4000.

### Data Availability

The mass spectrometry proteomics data have been deposited to the ProteomeXchange Consortium via the PRIDE³⁶ partner repository with the dataset identifier PXD025012. ChIP-sequencing data can be found under the reference number GSE169317 in the GEO database.

TABLE 1
Primers and oligonucleotides
Cloning primers and gBlocks

| | |
|---|---|
| TurboEcoRI-F | CCGAATTCGCTAGCAAAGACAATACTGTG |
| TurboBamHIfrag-R | GGCCAGCTCCACCAGGATCCCTGCCAGCT |
| TurboBamHIfrag-F | AGCTGGCAGGGATCCTGGTGGAGCTGGCC |
| TurboBamHI-R | CCGGATCCTCAGTCGGCCCTGCTGAATTCCTTTTCG |
| 6xHistagHinDIII-F | AGCTTCATGCATCATCACCACCACCAT |
| 6xHistagHinDIII-R | AGCTATGGTGGTGGTGATGATGCATGA |
| HA1emerinMluI-Fw | CCGACGCGTTAGTGGCGTCCGGGCTCGCAGTAC |
| HA1emerinNcoI-Rv | GGCCATGGGGCCTACCAAAACCGGCCGAGCAACG |
| HA2emerinHindIII-Fw | CCAAGCTTGCATGGACAACTACGCAGATCTTTCGGA |
| HA2emerinAscI-Rv | GAAGGCGCGCCCCAACCCCTGCCTTACCCTTGCTCTG |
| gRNAEmerinFw2 | CACCGCGCCCACGCCCGAGTCCGCC |
| gRNAEmerinRv2 | AAACGGCGGACTCGGGCGTGGGCGC |
| HA2FLYWCH1NotI-Fw | CCGCGGCCGCACAGGCCCTGGGTCCCGGGATGCCCCT |
| HA2FLYWCH1AscI-Rv | CCGGCGCGCCGGAACGGGAAGGCTAGGTTAGCTAA |
| TurboNheIfrag-F | GCAGGATAGAAAGCTAGCAGGCATCCTGG |
| TurboNheIfrag-R | CCAGGATGCCTGCTAGCTTTCTATCCTGC |
| TurboNotI-R | CCGCGGCCGCGTCGGCCCTGCTGAATTCCTTTTC |
| MiniTurbogoodNheI-F | GACTACGCAGCTAGCATCCCGCTGCTG |
| gRNAFLYWCH1Fw | CACCGGGGTGCTGAGCGTGGCCTGA |
| gRNAFLYWCH1Rv | AAACTCAGGCCACGCTCAGCACCCC |
| FLYWCH1EcorI-Fw | CCGAATTCGGATGCCCCTGCCCGAGCCCAGCGAG |
| FLYWCH1KpnI-Rv | CCGGTACCTCACTGGGACTCGCCATCCAGTCTGAC |
| 3xV5 | GCTCCGCGGCCGCAATGATCCCTAACCCTCTCCTCGGTCTCGATATTC |
| | CAAATCCCTTACTCGGCCTAGACATACCGAACCCATTGCTTGGACTGG |
| | ATGGCGGGGGAGGTTCTGGTGGGGGCGGATCAGCGGCCGCACAGGC |
| HA1FLYWCH1 | CCCATATGGTATCTCCTGAAGTGGGGGTGGCCTGGGGCCTCCAGGCTA |
| | GGATAGGGACTGGGTCTGCCTGAGCCCAGAAAGGGCTTTCTGCCTCCC |
| | AGGCCGTGATGTGGTGCCGTCCTCTGGAAAGGGCTCTGCGTACCAGGC |
| | CCACATGCCTGAGACAGGTGCCTGGCGTCCTGGTGAAGCACATGGTTT |
| | CCAAAAAGGTCCTGACAAGTCCCTGGGCATCTGAGCAGAAACGGGTCT |
| | CAGCTTCCCGCCTCTTCAGCCTCAAGTGTATCACAGGTGTCTGTGCCT |
| | CACTATCCTCGTCTATAACTGGGTCAATAAGAACGTTCTGCCGTCATA |
| | ATGTAGGGACTGGAGGGGGGACTGGAAACGCGGGTGGAAACCCTTCGT |
| | CCGTGCTCAGCATGTGGCTGAGCTCTTAGTCCATTAGTCACTCTGATG |
| | GCCCCTTGTGGAGGGTGTTTATTGTGCACATTTTCCAGATGGGGAAAG |
| | TGGGGTTGGATGGGGCTGACGTACCTAAGGCTGCCAGGAGAGGAACTG |
| | GCAGAGCAGAAGCCTAGTTCCATCAGGCCCCCGGCCAGTCTTGGCCCC |
| | AGCGAGCAGTGAGGCAGCCCCATCTGCCCCACCTCCCTCCCAGATCCA |
| | CGTCTAGAGTCCCCCAAGGGCTTCCACCACTGACGGGATTTTGCTTCC |
| | TTCCTTAGGACGGAACCACTGCACTCCAGGTTCCTTGCTGGGTGCTGA |
| | GCGTGGCCTGACCATGGGG |

### Results

Off the Shelf Proximity Labeling in Fixed Cells using ProtA-Turbo Enzyme

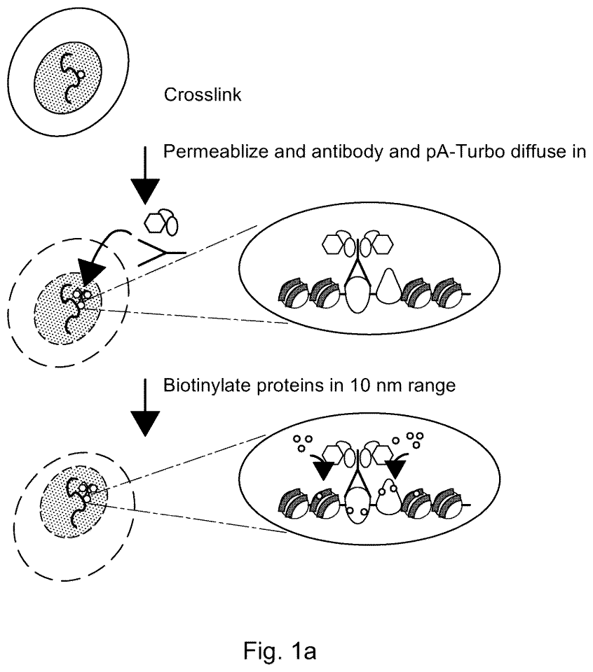

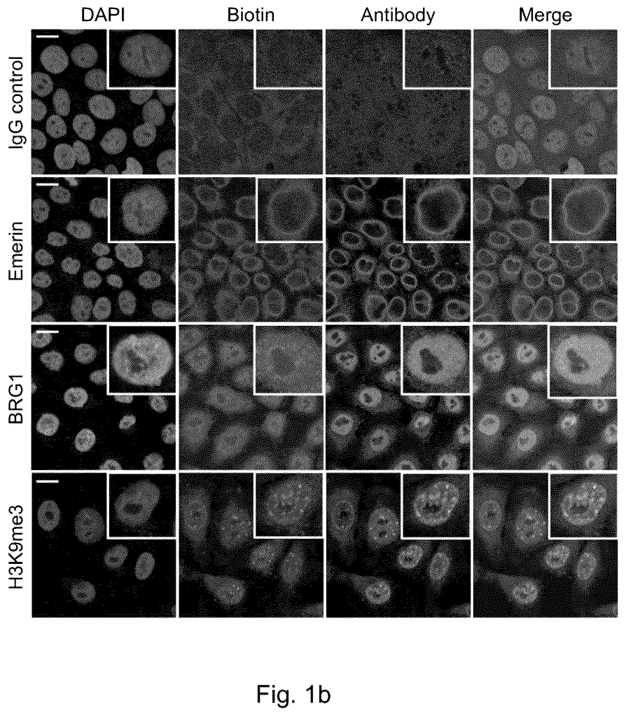

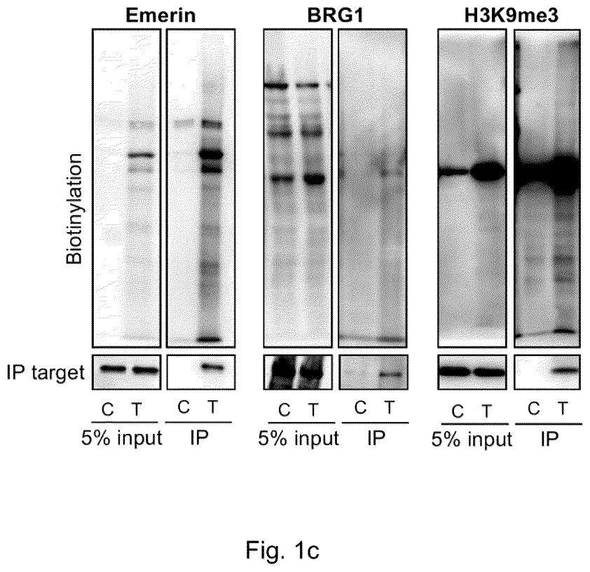

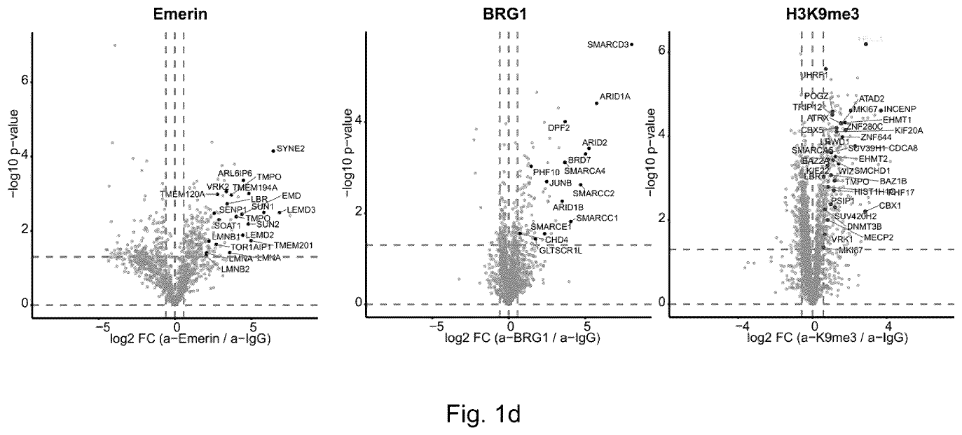

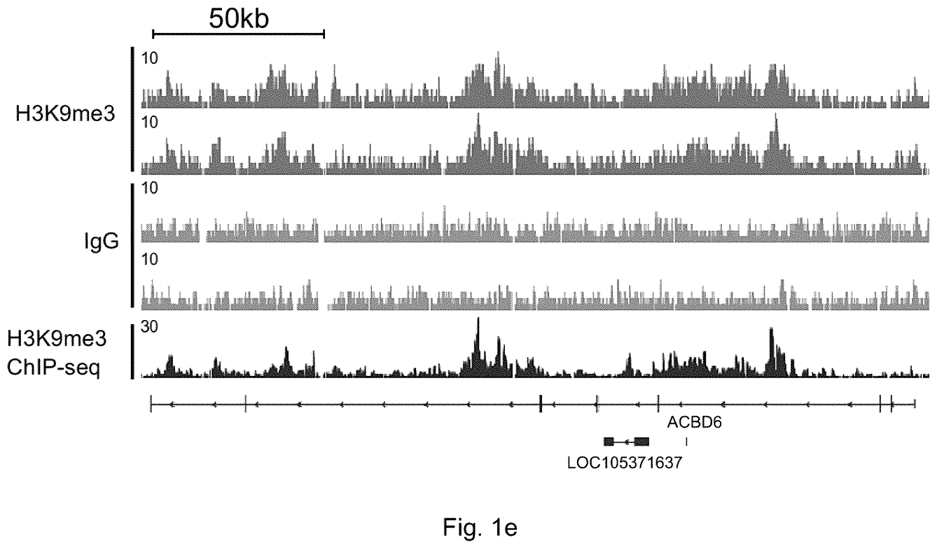

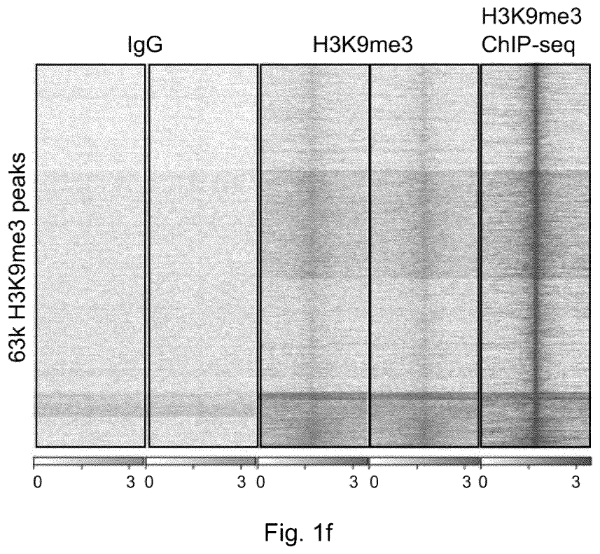

With the aim to design an ‘off the shelf’ proximity biotinylation enzyme that does not require genetic manipulation or transfection of target cells, we generated a construct consisting of Protein A fused to the recently developed proximity biotinylation enzyme TurboID. The TurboID enzyme is a much faster, modified version of the BioID proximity biotinylation enzyme which can biotinylate proximal proteins inside cells in minutes. This ProtA-Turbo fusion protein was expressed and purified as a His-tagged fusion protein from bacteria (data not shown). Initial activity tests revealed that the purified ProtA-Turbo enzyme efficiently triggers protein biotinylation in bacterial cells in vivo (data not shown). Furthermore, the ProtA-Turbo enzyme also triggers protein biotinylation when added to mammalian cell extracts (data not shown). We first developed a workflow for the ProtA-Turbo enzyme in fixed cells (FIG. 1a). After formaldehyde-based fixation, cells are permeabilized followed by subsequent addition of a primary antibody and the ProtA-Turbo enzyme. An isotype control IgG antibody is used as a negative control. Following several wash steps to remove unbound antibody and enzyme, addition of exogenous biotin triggers bait-proximal protein biotinylation. These biotinylated proteins can subsequently be enriched from crude cell lysates using streptavidin-based affinity enrichment and identified using quantitative LC-MS.

To benchmark this method, we made use of antibodies targeting various well-characterized nuclear baits: Emerin, BRG1 and a histone modification, H3K9me3. Immunofluorescence-based analysis revealed that ProtA-Turbo mediated biotinylation using these antibodies is induced in a pattern that is in agreement with the known nuclear localization of the respective baits. For example, the Emerin antibody, which resides in the nuclear envelop, triggers ProtA-Turbo mediated biotinylation in the nuclear rim, whereas the H3K9me3 antibody results in a punctuated biotinylation signal in the nucleus that is reminiscent of the DAPI-dense chromocenters in mammalian nuclei, which are enriched for H3K9me3. In contrast, the BRG1 antibody, which targets the large multi-subunit Swi/Snf complex, results in a more diffuse nuclear staining (FIG. 1b). Streptavidin-based affinity enrichment of ProtA-Turbo targeted cells with these antibodies revealed efficient enrichment of the targeted baits and associated protein biotinylation (FIG. 1c). ProtA-turbo experiments followed by streptavidin-based affinity enrichments were then performed in triplicate with the respective baits and negative IgG controls followed by mass spectrometry analyses. As shown in FIG. 1d, each of the three bait antibodies in combination with the ProtA-Turbo enzyme resulted in a cluster of specifically enriched biotinylated proteins, including many positive control proteins. For example, various nuclear lamina-associated proteins are enriched in the Emerin ProtA-turbo experiments, including TMPO, SUN2 and LMNB2. As expected, the BRG1 antibody in combination with the ProtA-Turbo enzyme induced enrichment of numerous Swi/Snf complex subunits such as ARID1A, BRD7 and SMARCC1. Finally, the H3K9me3 antibody resulted in specific enrichment of the H3K9 methyltransferases EHMT1/2 and Suv39H1, various known H3K9me3 reader proteins (i.e. CBX5, CBX1 and UHRF1), as well as centromere-associated proteins (INCENP, CDCA8). Centromeres are known to be enriched for the H3K9me3 modification. GO term enrichment analysis for the different affinity purifications are in agreement with this: membrane and nuclear pore associated GO terms are enriched in the Emerin experiment, Swi/snf subunits in the BRG1-ProtA-Turbo enrichment and heterochromatin and condensed chromosome terms are enriched in the H3K9me3 experiment (data not shown). As a control experiment, we generated a knock-in cell line in which we fused Emerin to TurboID. Immunofluorescence experiments revealed biotinylation signals in this cell line at the nuclear rim (data not shown), similar to what we observed when targeting ProtA-Turbo with an Emerin antibody. Furthermore, biotin purifications of crude lysates from the Turbo-Emerin knock-in cells revealed specific enrichment of many known nuclear lamina associated proteins, many of which were also retrieved in the protA-Turbo Emerin targeting experiment (data not shown). GO term enrichment analysis of the Emerin proximity labeling experiments performed in this study are also in excellent agreement with previous interaction proteomics experiments targeting the nuclear lamina (data not shown).

To further illustrate the ability of our method to trigger antibody-dependent, bait-specific biotinylation, we performed biotin ChIP-seq using streptavidin-conjugated beads after H3K9me3 or IgG targeting with ProtA-Turbo. This experiment revealed biotinylated chromatin patterns that are significantly overlapping with previously generated H3K9me3 ChIP-seq data in the same target cells (FIG. 1e,f). Furthermore, comparison of our ProtA-Turbo-H3K9me3 interactome with a recently published proteomics method called ChromID, in which proximity biotinylation is targeted to chromatin modifications using modification-specific reader domains fused to a proximity biotinylation enzyme, revealed that many H3K9me3-proximal proteins are identified using both methodologies (data not shown).

In summary, these experiments clearly validate and illustrate the value of the ProtA-turbo enzyme as an off the shelf proximity biotinylation enzyme in the absence of genetic manipulation or transfection of target cells, at least in a workflow with fixed cells.

Off the Shelf Proximity Labeling in Non-Fixed Cells using ProtA-Turbo

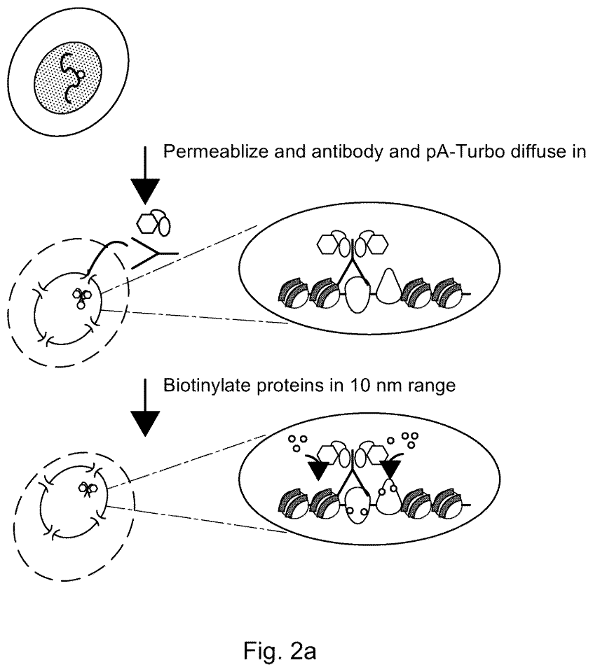

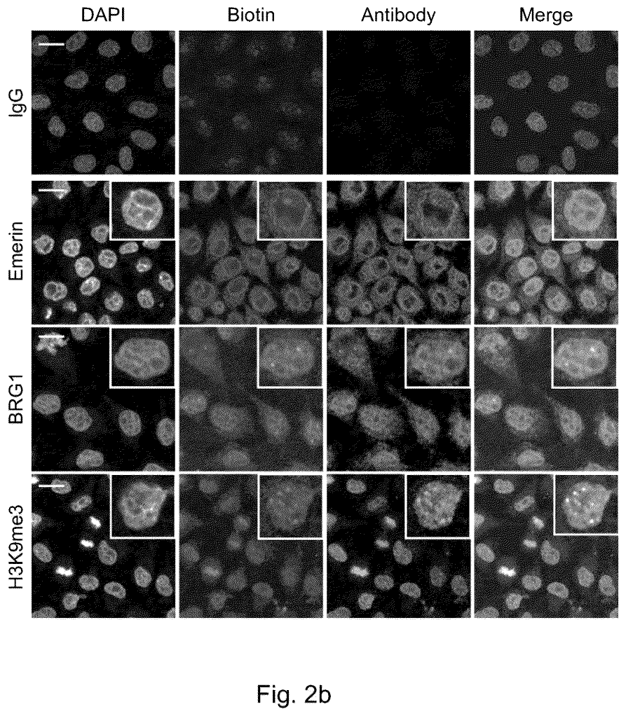

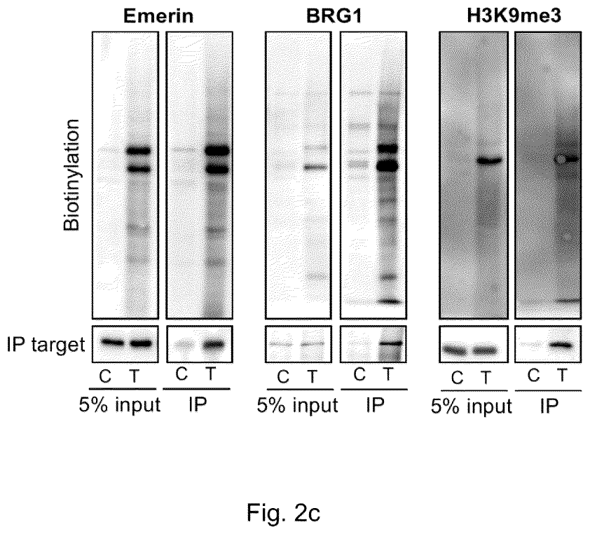

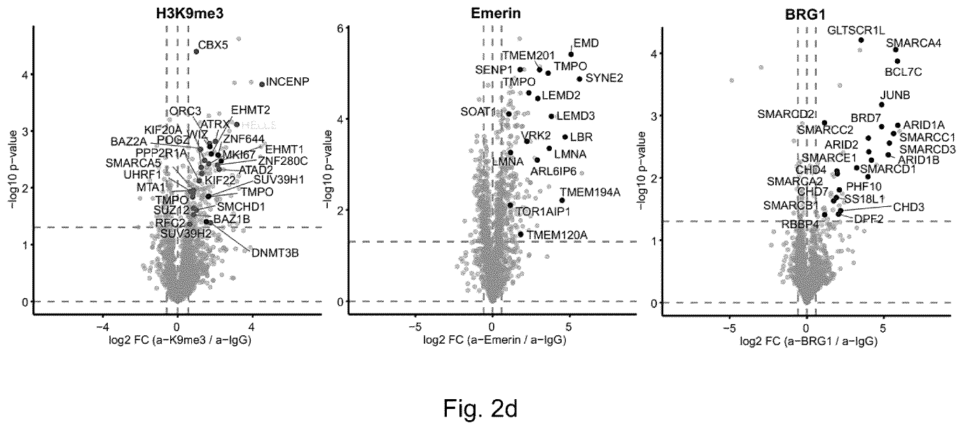

Next, to further expand the applicability of the ProtA-Turbo enzyme, we set out to establish a workflow in which the ProtA-Turbo enzyme is targeted to baits of interest in non-fixed cells (FIG. 2a), using the same antibodies that were also used in fixed cells (Emerin, BRG1 and H3K9me3). Advantages of the omission of crosslinking are the availability of certain downstream applications which are not possible on cross-linked material. Furthermore, the required laboratory material for the workflow using non-fixed cells is restricted to a bench-top centrifuge and a shaker. To avoid disruption of membrane structures during the procedure, we used the mild detergent digitonin for cell permeabilization. Digitonin is also used in recently developed epigenome profiling tools such as CUT & RUN¹⁰. Immunofluorescence experiments in non-fixed cells revealed expected biotinylation patterns using bait-specific antibodies, although not as clear as observed when using fixed cells (FIG. 2b). Subsequent streptavidin affinity enrichment of crude lysates revealed bait-specific interactomes of high quality, with many expected proteins and enriched GO terms (FIG. 2c-d).

Having established efficient off the shelf proximity biotinylation strategies for the ProtA-Turbo enzyme in fixed and non-fixed cells, we aimed to further demonstrate the broad applicability of the ProtA-Turbo workflow. To this end, we performed H3K9me3 targeting experiments in a breast cancer cell line (MCF7 cells) and in myeloid leukemia cells (U937) (data not shown). These experiments, using fixed and non-fixed cells, respectively, revealed a range of known heterochromatin proteins, as well as many overlapping but also distinct H3K9me3 proximal proteins in both cell lines. Finally, we performed ProtA-Turbo Emerin targeting experiments in primary material, namely low passage primary human fibroblasts. This revealed correct biotinylation targeting of the nuclear envelope as assessed using immunofluorescence (data not shown). Subsequent mass spectrometry-based analyses revealed many known nuclear envelope components, thus demonstrating the applicability of the ProtA-turbo enzyme to identify bait-proximal proteins in non-fixed, primary cells (data not shown).

### FLYWCH1 Localizes to Centromeric, H3K9me3-Marked Chromatin

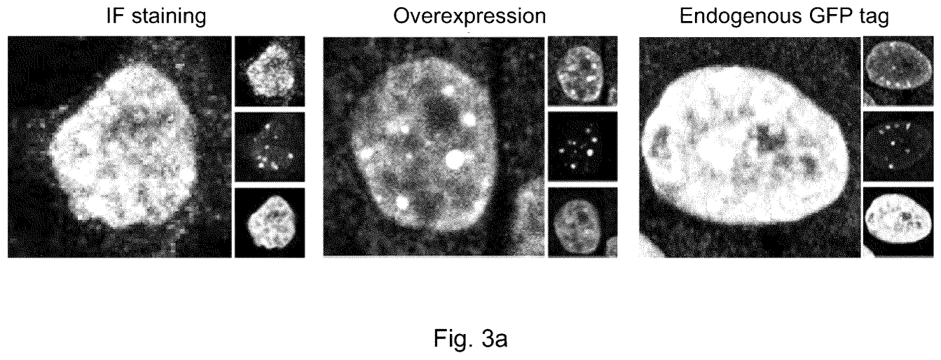

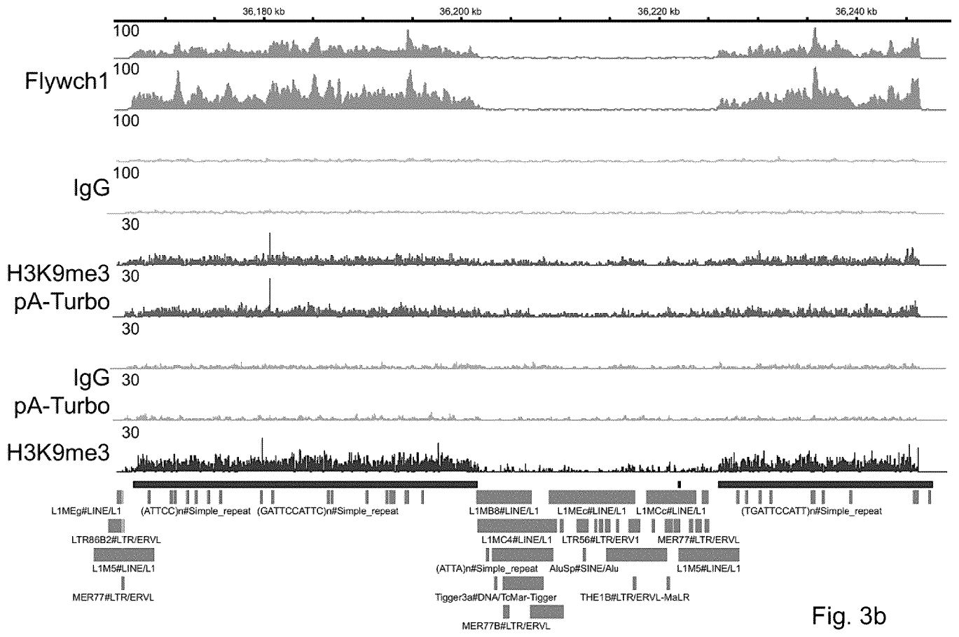

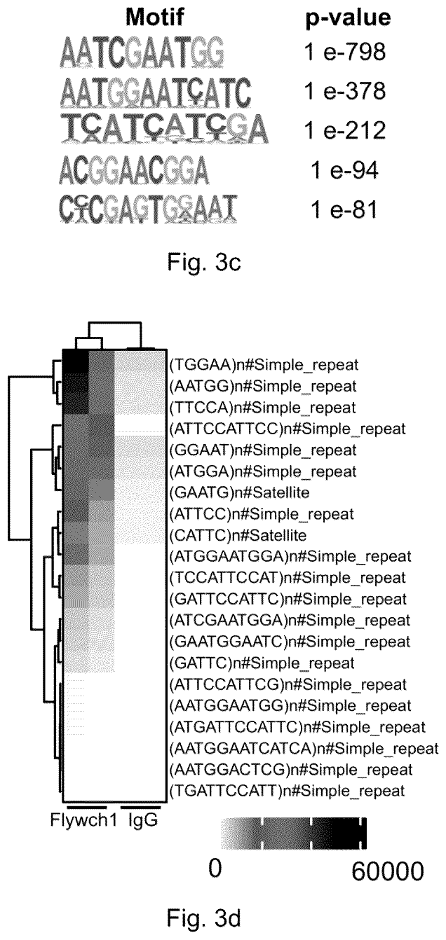

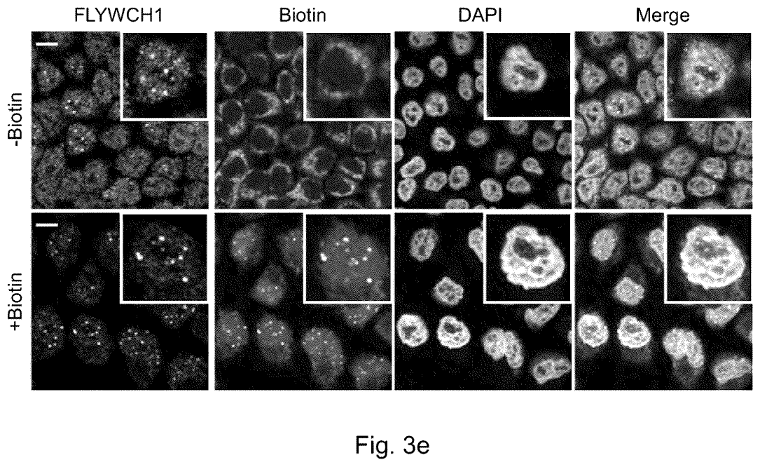

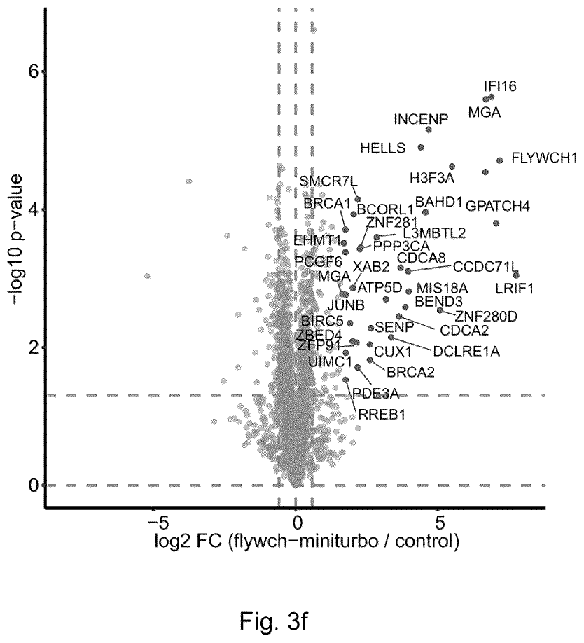

The ProtA-Turbo proximity biotinylation experiments revealed many known but also many previously unknown proximal proteins for the baits investigated. As an example, in various ProtA-Turbo H3K9me3 targeting experiments a new H3K9me3-proximal protein called FLYWCH1 was uncovered (data not shown). FLYWCH1 contains 5 so-called FLYWCH-type Zinc fingers and has previously been identified as a regulator of beta-catenin signaling and has been linked to various malignancies. Furthermore, a homozygous Flywch1 deletion is embryonic lethal in mice. To study this protein in more detail, we transiently overexpressed GFP-FLYWCH1 and created cell lines where FLYWCH1 is tagged endogenously with GFP and V5 (data not shown). Immunofluorescence experiments revealed a strong overlap between FLYWCH1 and H3K9me3 in mammalian nuclei, consistent with our ProtA-Turbo H3K9me3 proteomics experiments (FIG. 3a). Next, we performed ChIP-seq analysis for FLYWCH1 in duplicate using the cell line in which endogenous FLYWCH1 is tagged with GFP and a V5 tag (data not shown). These experiments revealed a significant genome-wide overlap between H3K9me3 and FLYWCH1 (FIG. 3b). DNA motif analyses amongst 451 high confidence FLYWCH1 binding sites in the genome revealed a strong enrichment of simple repeats (FIG. 3c,d). Interestingly, many strong FLYWCH1 binding sites in the genome are localized at centromeric heterochromatin, which is known to be enriched for H3K9me3 (data not shown). To gain further insights into the function of the FLYWCH1 protein, we generated a knock-in cell line in which FLYWCH1 is tagged with a proximity labeling enzyme mini-Turbo, which is a smaller and less active version of the TurboID enzyme. Immunofluorescence experiments revealed that nuclear biotinylation in this FLYWCH1-miniTurbo cell line is localized in punctuated foci, which is consistent with the H3K9me3 staining pattern in mammalian cells (FIG. 3e). Subsequent streptavidin-based affinity enrichment experiments from miniTurbo-FLYWCH1 cells revealed numerous FLYWCH1 proximal proteins (FIG. 3f). Interestingly, these FLYWCH1-proximal proteins include the H3K9me3 methyltransferase EHMT1, HP1 interactor MGA, subunits of the chromosomal passenger complex (INCENP, CDCA8) and Polycomb proteins. In summary, these experiments uncovered FLYWCH1 as a new H3K9me3 proximal protein that localizes to centromeric heterochromatin, together with many additional H3K9me3 associated proteins.

### BIBLIOGRAPHY

1. Smits, A. H. & Vermeulen, M. Characterizing Protein-Protein Interactions Using Mass Spectrometry: Challenges and Opportunities. Trends Biotechnol. 34, 825-834 (2016).
2. Samavarchi-Tehrani, P., Samson, R. & Gingras, A. C. Proximity dependent biotinylation: Key enzymes and adaptation to proteomics approaches. Mol. Cell. Proteomics 19, 757-773 (2020).
3. Roux, K. J., Kim, D. I., Raida, M. & Burke, B. A promiscuous biotin ligase fusion protein identifies proximal and interacting proteins in mammalian cells. J. Cell Biol. 196, 801-810 (2012).
4. Qin, W., Cho, K. F., Cavanagh, P. E. & Ting, A. Y. Deciphering molecular interactions by proximity labeling. Nat. Methods 18, (2021).
5. Go, C. D. et al. A proximity biotinylation map of a human cell. bioRxiv (2019). doi:doi:https://doi.org/10.1101/796391
6. van Mierlo, G. & Vermeulen, M. Chromatin proteomics to study epigenetics—challenges and opportunities. Mol. Cell. Proteomics 20, (2021).
7. Moquin, D. M. et al. Localized protein biotinylation at dna damage sites identifies zpet, a repressor of homologous recombination. Genes Dev. 33, 253 (2019).
8. Hung, V. et al. Proteomic mapping of cytosol-facing outer mitochondrial and ER membranes in living human cells by proximity biotinylation. Elife 6, 1-39 (2017).
10. Skene, P. J. & Henikoff, S. An efficient targeted nuclease strategy for high-resolution mapping of DNA binding sites. Elife 6, 1-35 (2017).
14. Pchelintsev, N. A., Adams, P. D. & Nelson, D. M. Critical parameters for efficient sonication and improved chromatin immunoprecipitation of high molecular weight proteins. PLoS One 11, 1-11 (2016).
15. Bar, D. Z. et al. Biotinylation by antibody recognition-a method for proximity labeling. Nat. Methods 15, 127-133 (2018).
16. Rees, J. S., Li, X. W., Perrett, S., Lilley, K. S. & Jackson, A. P. Selective proteomic proximity labeling assay using tyramide (SPPLAT): A quantitative method for the proteomic analysis of localized membrane-bound protein clusters. Current Protocols in Protein Science 80, 19.27.1-19.27.18 (2015).
17. Makowski, M. M., Willems, E., Jansen, P. W. T. C. & Vermeulen, M. Cross-linking immunoprecipitation-MS (xIP-MS): Topological Analysis of Chromatin-associated Protein Complexes Using Single Affinity Purification. Mol. Cell. Proteomics 15, 854-865 (2016).
18. Yu, C. & Huang, L. Cross-linking Mass Spectrometry (XL-MS): and emerging technology for interactomics and structural biology. Anal Chem 90, 144-165 (2018).
19. Villaseñor, R. et al. ChromID identifies the protein interactome at chromatin marks. Nat. Biotechnol. 38, 728-736 (2020).
21. Rappsilber, J., Mann, M. & Ishihama, Y. Protocol for micro-purification, enrichment, pre-fractionation and storage of peptides for proteomics using StageTips. Nature Protocols 2, 1896-1906 (2007).
22. Kloet, S. L. et al. The dynamic interactome and genomic targets of Polycomb complexes during stem-cell differentiation. Nat. Struct. Mol. Biol. 23, 682-690 (2016).
23. Furlan, C. et al. Miniaturised interaction proteomics on a microfluidic platform with ultra-low input requirements. Nat. Commun. 10, 1-8 (2019).
24. Saksouk, N. et al. Redundant Mechanisms to Form Silent Chromatin at Pericentromeric Regions Rely on BEND3 and DNA Methylation. Mol. Cell 56, 580-594 (2014).
25. Yu, D. et al. High-resolution genome-wide scan of genes, gene-networks and cellular systems impacting the yeast ionome. BMC Genomics 13, (2012).
26. Cutler, J. A. et al. Mapping the micro-proteome of the nuclear lamina and lamin associated domains. bioRxiv 1-36 (2019). doi:10.1101/828210
27. Chojnowski, A. et al. 2C-BioID: An Advanced Two Component BioID System for Precision Mapping of Protein Interactomes. iScience 10, 40-52 (2018).
28. Shannon, P. et al. Cytoscape: A Software Environment for Integrated Models of Biomolecular Interaction Networks. Genome Res. 13, 2498-2504 (2021).
29. Li, H. Aligning sequence reads, clone sequences and assembly contigs with BWA-MEM. airXiv (2013).
30. Timms, R. T., Tchasovnikarova, I. A., Antrobus, R., Dougan, G. & Lehner, P. J. ATF7IP-Mediated Stabilization of the Histone Methyltransferase SETDB1 Is Essential for Heterochromatin Formation by the HUSH Complex. Cell Rep. 17, 653-659 (2016).
31. Zhang, Y. et al. Model-based analysis of ChIP-Seq (MACS). Genome Biol. 9, (2008).
32. Georgiou, G. & van Heeringen, S. J. Fluff: Exploratory analysis and visualization of high-throughput sequencing data. PeerJ (2016). doi:10.7717/peerj.2209
33. Heinz, S. et al. Simple Combinations of Lineage-Determining Transcription Factors Prime cis-Regulatory Elements Required for Macrophage and B Cell Identities. Mol. Cell 38, 576-589 (2010).
34. Gel, B. & Serra, E. KaryoploteR: An R/Bioconductor package to plot customizable genomes displaying arbitrary data. Bioinformatics 33, 3088-3090 (2017).
36. Perez-Riverol, Y. et al. The PRIDE database and related tools and resources in 2019: Improving support for quantification data. Nucleic Acids Res. 47, D442-D450 (2019).

1. A fusion polypeptide comprising a biotin ligase enzyme fused to an immunoglobulin-binding bacterial protein, preferably wherein the immunoglobulin-binding bacterial protein is selected from Protein A, Protein G, Protein A/G and Protein L.

2. The fusion polypeptide according to claim 1, wherein the biotin ligase enzyme has proximity-dependent biotinylation activity.

3. A composition comprising the fusion polypeptide according to claim 1 and further comprising an immunoglobulin, preferably an antibody, more preferably a monoclonal antibody, wherein said antibody targets the fusion polypeptide to a subcellular region of interest.

4. A complex comprising the fusion polypeptide according to claim 1 complexed to an immunoglobulin, preferably an antibody, more preferably a monoclonal antibody, wherein said antibody targets the complexed fusion polypeptide to a subcellular region or protein of interest.

5. The composition according to claim 3, wherein the antibody is an IgG antibody, and the immunoglobulin-binding bacterial protein binds to the Fc region of said IgG.

6. A method for biotinylating a protein of interest in a cell, a subcellular region or a sample of interest, the method comprising:
a) contacting the sample with the composition according to claim 3; and
b) adding biotin or a derivative thereof and ATP to the sample, wherein the biotin ligase biotinylates the protein.

7. A method of proximity labeling of proteins in a cell, the method comprising:
a) introducing the composition according to claim 3 into a cell, wherein the fusion polypeptide comprising the biotin ligase is targeted to a subcellular region of interest; and
b) contacting the cell with biotin or a derivative thereof and ATP, wherein proteins in proximity to the biotin ligase are biotinylated.

8. A kit for biotinylating a protein of interest in a cell, a subcellular region or a sample of interest, comprising:
a fusion polypeptide according to claim 1, and
an immunoglobulin, preferably an antibody, more preferably a monoclonal antibody, preferably an antibody to which the immunoglobulin-binding bacterial protein binds.

9. A kit according to claim 8, wherein said immunoglobulin targets the fusion polypeptide to a protein or subcellular region of interest.

10. A kit according to claim 8, further comprising biotin, or a derivative thereof, and ATP.


SEQUENCE LISTING (54 sequences)

SEQ ID NO: 1 - protein, 321 aa, Artificial Sequence
  wild-type biotin ligase enzyme
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACVAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEK

SEQ ID NO: 2 - protein, 257 aa, Artificial Sequence
  sequence A
IPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAAMLIRELRAALELFEQEGLAPYLSRWEKLDNFINRPVKLIIGDKEIFGISRGIDKQGALLLEQDGVIKPWMGGEISLRSAEK

SEQ ID NO: 3 - protein, 319 aa, Artificial Sequence
  sequence B
KDNTVPLKLIALLANGEFHSGEQLGETLGMSRAAINKHIQTLRDWGVDVFTVPGKGYSLPEPIPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAATLIRELRAALELFEQEGLAPYLPRWEKLDNFINRPVKLIIGDKEIFGISRGIDKQGALLLEQDGVIKPWMGGEISLRSAEK

SEQ ID NO: 4 - protein, 508 aa, Staphylococcus aureus
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNKFNKDQQSAFYEILNMPNLNEEQRNGFIQSLKDDPSQSTNVLGEAKKLNESQAPKADNNFNKEQQNAFYEILNMPNLNEEQRNGFIQSLKDDPSQSANLLAEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDGNKPGKEDGNKPGKEDNKKPGKEDGNKPGKEDNKKPGKEDGNKPGKEDGNKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 5 - protein, 441 aa, Artificial Sequence
  Immunoglobulin G-binding protein A from Staphylococcus aureus (UniProtKB - P38507) after processing (1-36 and 478-508 are removed)
AQHDEAQQNAFYQVLNMPNLNADQRNGFIQSLKDDPSQSANVLGEAQKLNDSQAPKADAQQNKFNKDQQSAFYEILNMPNLNEEQRNGFIQSLKDDPSQSTNVLGEAKKLNESQAPKADNNFNKEQQNAFYEILNMPNLNEEQRNGFIQSLKDDPSQSANLLAEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDGNKPGKEDGNKPGKEDNKKPGKEDGNKPGKEDNKKPGKEDGNKPGKEDGNKPGKEDGNKPGKEDGNKPGKEDGNGVHVVKPGDTVNDIAKANGTTADKIAADNKLADKNMIKPGQELVVDKKQPANHADANKAQALPET

SEQ ID NO: 6 - protein, 593 aa, Streptococcus sp.
  MISC_FEATURE (1)..(593): Streptococcus sp. group G
MEKEKKVKYFLRKSAFGLASVSAAFLVGSTVFAVDSPIEDTPIIRNGGELTNLLGNSETTLALRNEESATADLTAAAVADTVAAAAAENAGAAAWEAAAAADALAKAKADALKEFNKYGVSDYYKNLINNAKTVEGVKDLQAQVVESAKKARISEATDGLSDFLKSQTPAEDTVKSIELAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPKTDTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEMVTEVPGDAPTEPEKPEASIPLVPLTPATPIAKDDAKKDDTKKEDAKKPEAKKEDAKKAETLPTTGEGSNPFFTAAALAVMAGAGALAVASKRKED

SEQ ID NO: 7 - protein, 529 aa, Artificial Sequence
  Immunoglobulin G-binding protein G from Streptococcus sp. group G (UniProtKB - P19909) after processing (1-33 and 563-593 are removed)
VDSPIEDTPIIRNGGELTNLLGNSETTLALRNEESATADLTAAAVADTVAAAAAENAGAAAWEAAAAADALAKAKADALKEFNKYGVSDYYKNLINNAKTVEGVKDLQAQVVESAKKARISEATDGLSDFLKSQTPAEDTVKSIELAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPKTDTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEMVTEVPGDAPTEPEKPEASIPLVPLTPATPIAKDDAKKDDTKKEDAKKPEAKKEDAKKAETLPTT

SEQ ID NO: 8 - DNA, 20 bp, Artificial Sequence
  gRNA
gggtgctgagcgtggcctga

SEQ ID NO: 9 - DNA, 20 bp, Artificial Sequence
  gRNA
cgcccacgcccgagtccgcc

SEQ ID NO: 10 - DNA, 29 bp, Artificial Sequence
  TurboEcoRI-F
ccgaattcgctagcaaagacaatactgtg

SEQ ID NO: 11 - DNA, 29 bp, Artificial Sequence
  TurboBamHIfrag-R
ggccagctccaccaggatccctgccagct

SEQ ID NO: 12 - DNA, 29 bp, Artificial Sequence
  TurboBamHIfrag-F
agctggcagggatcctggtggagctggcc

SEQ ID NO: 13 - DNA, 36 bp, Artificial Sequence
  TurboBamHI-R
ccggatcctcagtcggccctgctgaattccttttcg

SEQ ID NO: 14 - DNA, 27 bp, Artificial Sequence
  6xHistagHinDIII-F
agcttcatgcatcatcaccaccaccat

SEQ ID NO: 15 - DNA, 27 bp, Artificial Sequence
  6xHistagHinDIII-R
agctatggtggtggtgatgatgcatga

SEQ ID NO: 16 - DNA, 33 bp, Artificial Sequence
  HA1emerinMluI-Fw
ccgacgcgttagtggcgtccgggctcgcagtac

SEQ ID NO: 17 - DNA, 34 bp, Artificial Sequence
  HA1emerinNcoI-Rv
ggccatggggcctaccaaaaccggccgagcaacg

SEQ ID NO: 18 - DNA, 36 bp, Artificial Sequence
  HA2emerinHindIII-Fw
ccaagcttgcatggacaactacgcagatctttcgga

SEQ ID NO: 19 - DNA, 37 bp, Artificial Sequence
  HA2emerinAscI-Rv
gaaggcgcgccccaacccctgccttacccttgctctg

SEQ ID NO: 20 - DNA, 25 bp, Artificial Sequence
  gRNAEmerinFw2
caccgcgcccacgcccgagtccgcc

SEQ ID NO: 21 - DNA, 25 bp, Artificial Sequence
  gRNAEmerinRv2
aaacggcggactcgggcgtgggcgc

SEQ ID NO: 22 - DNA, 37 bp, Artificial Sequence
  HA2FLYWCH1NotI-Fw
ccgcggccgcacaggccctgggtcccgggatgcccct

SEQ ID NO: 23 - DNA, 35 bp, Artificial Sequence
  HA2FLYWCH1AscI-Rv
ccggcgcgccggaacgggaaggctaggttagctaa

SEQ ID NO: 24 - DNA, 29 bp, Artificial Sequence
  TurboNheIfrag-F
gcaggatagaaagctagcaggcatcctgg

SEQ ID NO: 25 - DNA, 29 bp, Artificial Sequence
  TurboNheIfrag-R
ccaggatgcctgctagctttctatcctgc

SEQ ID NO: 26 - DNA, 34 bp, Artificial Sequence
  TurboNotI-R
ccgcggccgcgtcggccctgctgaattccttttc

SEQ ID NO: 27 - DNA, 27 bp, Artificial Sequence
  MiniTurbogoodNheI-F
gactacgcagctagcatcccgctgctg

SEQ ID NO: 28 - DNA, 25 bp, Artificial Sequence
  gRNAFLYWCH1Fw
caccggggtgctgagcgtggcctga

SEQ ID NO: 29 - DNA, 25 bp, Artificial Sequence
  gRNAFLYWCH1Rv
aaactcaggccacgctcagcacccc

SEQ ID NO: 30 - DNA, 34 bp, Artificial Sequence
  FLYWCH1EcorI-Fw
ccgaattcggatgcccctgcccgagcccagcgag

SEQ ID NO: 31 - DNA, 35 bp, Artificial Sequence
  FLYWCH1KpnI-Rv
ccggtacctcactgggactcgccatccagtctgac

SEQ ID NO: 32 - DNA, 142 bp, Artificial Sequence
  3xV5
gctccgcggccgcaatgatccctaaccctctcctcggtctcgatattccaaatcccttactcggcctagacataccgaacccattgcttggactggatggcgggggaggttctggtgggggcggatcagcggccgcacaggc

SEQ ID NO: 33 - DNA, 739 bp, Artificial Sequence
  HA1FLYWCH1
cccatatggtatctcctgaagtgggggtggcctggggcctccaggctaggatagggactgggtctgcctgagcccagaaagggctttctgcctcccaggccgtgatgtggtgccgtcctctggaaagggctctgcgtaccaggcccacatgcctgagacaggtgcctggcgtcctggtgaagcacatggtttccaaaaaggtcctgacaagtccctgggcatctgagcagaaacgggtctcagcttcccgcctcttcagcctcaagtgtatcacaggtgtctgtgcctcactatcctcgtctataactgggtcaataagaacgttctgccgtcataatgtagggactggaggggggactggaaacgcgggtggaaacccttcgtccgtgctcagcatgtggctgagctcttagtccattagtcactctgatggccccttgtggagggtgtttattgtgcacattttccagatggggaaagtggggttggatggggctgacgtacctaaggctgccaggagaggaactggcagagcagaagcctagttccatcaggcccccggccagtcttggccccagcgagcagtgaggcagccccatctgccccacctccctcccagatccacgtctagagtcccccaagggcttccaccactgacgggattttgcttccttccttaggacggaaccactgcactccaggttccttgctgggtgctgagcgtggcctgaccatgggg

SEQ ID NO: 34 - DNA, 10 bp, Artificial Sequence
  enriched DNA motif
aatcgaatgg

SEQ ID NO: 35 - DNA, 12 bp, Artificial Sequence
  enriched DNA motif
aatggaatcatc

SEQ ID NO: 36 - DNA, 10 bp, Artificial Sequence
  enriched DNA motif
tcatcatcga

SEQ ID NO: 37 - DNA, 10 bp, Artificial Sequence
  enriched DNA motif
acggaacgga

SEQ ID NO: 38 - DNA, 12 bp, Artificial Sequence
  enriched DNA motif
cccgagtggaat

SEQ ID NO: 39 - DNA, 10 bp, Artificial Sequence
  ChIP-seq read
attccattcc

SEQ ID NO: 40 - DNA, 10 bp, Artificial Sequence
  ChIP-seq read
atggaatgga

SEQ ID NO: 41 - DNA, 10 bp, Artificial Sequence
  ChIP-seq read
tccattccat

SEQ ID NO: 42 - DNA, 10 bp, Artificial Sequence
  ChIP-seq read
gattccattc

SEQ ID NO: 43 - DNA, 10 bp, Artificial Sequence
  ChIP-seq read
atcgaatgga

SEQ ID NO: 44 - DNA, 10 bp, Artificial Sequence
  ChIP-seq read
gaatggaatc

SEQ ID NO: 45 - DNA, 10 bp, Artificial Sequence
  ChIP-seq read
attccattcg

SEQ ID NO: 46 - DNA, 10 bp, Artificial Sequence
  ChIP-seq read
aatggaatgg

SEQ ID NO: 47 - DNA, 12 bp, Artificial Sequence
  ChIP-seq read
atgattccattc

SEQ ID NO: 48 - DNA, 13 bp, Artificial Sequence
  ChIP-seq read
aatggaatcatca

SEQ ID NO: 49 - DNA, 10 bp, Artificial Sequence
  ChIP-seq read
aatggactcg

SEQ ID NO: 50 - DNA, 10 bp, Artificial Sequence
  ChIP-seq read
tgattccatt

SEQ ID NO: 51 - DNA, 330 bp, Artificial Sequence
  pK19-6xHis-ProtA Turbocomplete - Linker sequence
tggaattgtgagcggataacaatttcacacaggaaacagctatgaccatgattacgccaagcttccatgcatcatcaccaccaccatagcttcatgcatcatcaccaccaccatagcttcatgcatcatcaccaccaccatagcttcatgcatcatcaccaccaccatagcttaaaagatgacccaagccaaagtgctaacctattgtcagaagctaaaaagttaaatgaatctcaagcaccgaaagcggataacaaattcaacaaagaacaacaaaatgctttctatgaaatcttacatttacctaacttaaacgaagaacaacgcaat

SEQ ID NO: 52 - DNA, 1210 bp, Artificial Sequence
  pK19-6xHis-ProtA Turbocomplete - Linker sequence
agaacaacaaaatgctttctatgaaattttacatttacctaacttaactgaagaacaacgtaacggcttcatccaaagccttaaagacgatccttcagtgagcaaagaaattttagcagaagctaaaaagctaaacgatgctcaagcaccaaaagatgacgataaagaattcgctagcaaagacaatactgtgcctctgaagctgatcgctctcctggctaatggcgagttccatagtggcgaacagctgggagaaaccctgggcatgtccagggccgctatcaacaagcacattcagactctgcgcgactggggcgtggacgtgttcaccgtgcccggaaagggctactctctgcccgagcctatcccgctgctgaacgctaaacagattctgggacagctggacggcgggagcgtggcagtcctgcctgtggtcgactccaccaatcagtacctgctggatcgaatcggcgagctgaagagtggggatgcttgcattgcagaatatcagcaggcagggagaggaagcagagggaggaaatggttctctccttttggagctaacctgtacctgagtatgttttggcgcctgaagcggggaccagcagcaatcggcctgggcccggtcatcggaattgtcatggcagaagcgctgcgaaagctgggagcagacaaggtgcgagtcaaatggcccaatgacctgtatctgcaggatagaaagctggcagggatcctggtggagctggccggaataacaggcgatgctgcacagatcgtcattggcgccgggattaacgtggctatgaggcgcgtggaggaaagcgtggtcaatcagggctggatcacactgcaggaagcagggattaacctggacaggaatactctggccgctacgctgatccgagagctgcgggcagccctggaactgttcgagcaggaaggcctggctccatatctgccacggtgggagaagctggataacttcatcaatagacccgtgaagctgatcattggggacaaagagattttcgggattagccgggggattgataaacagggagccctgctgctggaacaggacggagttatcaaaccctggatgggcggagaaatcagtctgcggtctgccgaaaaggaattcagcagggcctaatgaggatccccgggtaccgagctcgaattaattcactggccgtcgttttacaacgtc

SEQ ID NO: 53 - protein, 87 aa, Artificial Sequence
  pK19-6xHis-ProtA Turbocomplete - Linker sequence
HHHHHHSFMHHHHHHSFMHHHHHHSFMHHHHHHSLKDDPSQSANLLSEAKKLNESQAPKADNKFNKEQQNAFYEILHLPNLNEEQRN

SEQ ID NO: 54 - protein, 383 aa, Artificial Sequence
  pK19-6xHis-ProtA Turbocomplete - Linker sequence
EQQNAFYEILHLPNLTEEQRNGFIQSLKDDPSVSKEILAEAKKLNDAQAPKDDDKEFASKDNTVPLKLIALLANGEFHSGEQLGETLGMSRAAINKHIQTLRDWGVDVFTVPGKGYSLPEPIPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAATLIRELRAALELFEQEGLAPYLPRWEKLDNFINRPVKLIIGDKEIFGISRGIDKQGALLLEQDGVIKPWMGGEISLRSAEKEFSRA